(19) United States
(12) Patent Application Publication
(10) Pub. No.: US 2024/0287139 A1
(43) Pub. Date: Aug. 29, 2024

(54) A NOVEL COMPOUND ACTING AGAINST A SELECT GROUP OF BACTERIA

(71) Applicant: Northeastern University, Boston, MA (US)

(72) Inventors: Kim Lewis, Newton, MA (US); Yu Imai, Boston, MA (US)

(21) Appl. No.: 18/571,666

(22) PCT Filed: Jun. 23, 2022
(86) PCT No.: PCT/US22/34703

§ 371 Date: Dec. 18, 2023

Related U.S. Application Data
(60) Provisional application No. 63/323,671, filed on Mar. 25, 2022.
(60) Provisional application No. 63/299,290, filed on Jan. 13, 2022.
(60) Provisional application No. 63/213,876, filed on Jun. 23, 2021.

(51) Int. Cl. C07K 11/02; A01N 63/50; A61K 38/00; A61K 38/15; A61K 45/06; A61P 31/06

(52) U.S. Cl. CPC C07K 11/02; A01N 63/50; A61K 38/15; A61K 45/06; A61P 31/06; A61K 38/00

(57) ABSTRACT

The present invention directed to a novel macrocyclic depsipeptide compound, its derivatives, and their pharmaceutically acceptable salts, having selective antibacterial activity against M. tuberculosis.

### CROSS-REFERENCE TO RELATED APPLICATIONS

This application claims the benefit of U.S. Provisional Patent Application Nos. 63/213,876 (filed on Jun. 23, 2021), 63/299,290 (filed on Jan. 13, 2022), and 63/323,671 (filed on Mar. 25, 2022), all of which are hereby incorporated by reference for all purposes as if fully set forth herein.

### GOVERNMENT FUNDING

This invention was made with government support under Grant Number P01AI118687 awarded by NIH National Institutes of Health. The government has certain rights in the invention.

### FIELD OF THE INVENTION

The present invention relates to novel macrocyclic depsipeptide compounds useful for the treatment of bacterial infections, particularly mycobacterial infections. The invention also relates to method of use of the compound for the treatment of mycobacterial infections such as those caused by Mycobacterium tuberculosis (M. tuberculosis).

### BACKGROUND

Mycobacterium is a genus of bacterium including pathogens responsible for tuberculosis (M. tuberculosis) and leprosy (M. leprae). Tuberculosis (TB), in particular—despite the availability of anti-TB drugs such as isoniazide and rifampin—is considered to be one of the world's deadliest diseases. Tuberculosis kills 1.5 million people every year and is a high-priority infectious disease. Since M. tuberculosis rapidly develops resistance against clinically important drugs, typical treatment involves a 6-month therapy with a cocktail of four antibiotics: rifampicin, isoniazid, ethambutol and pyrazinamide. Long-term administration of rifampicin destructs human gut microbiome, and may ultimately generate undesired antibiotic-resistant mutants; therefore, a novel antibiotic that exhibits selective activity against M. tuberculosis is much needed.

### SUMMARY OF THE INVENTION

In various embodiments, the present invention is directed to a novel macrocyclic depsipeptide compound having selective antibacterial activity against M. tuberculosis. The compound and its derivatives, and their pharmaceutically acceptable salts, can be useful, for example, for the treatment of bacterial infections (e.g., mycobacterial infections). More particularly, embodiments of the present invention include compounds represented by Formula I:

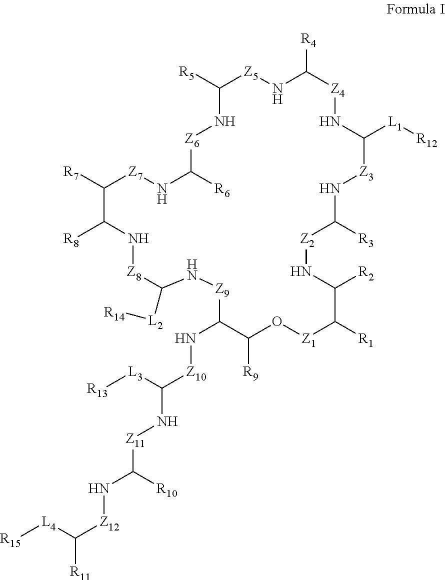

including a pharmaceutically acceptable salt, solvate or stereoisomer thereof:
wherein, in Formula I,
R₁ to R₁₀ are each independently selected from the group consisting of hydrogen, alkyl, alkenyl, alkynyl, hydroxyl, hydroxyalkyl, halogen, —CN, —O-alkyl, —C(O)-alkyl, —C(O)O-alkyl, —C(O)OH, —C(O)NH₂, —C(O)NH-alkyl, —NH₂, —NO₂, —CF₃, —NH-alkyl, —N— (alkyl)₂, —NHC(O)-alkyl, -aryl, -alkylaryl, alkylheteroaryl, wherein said alkyl, alkenyl, alkynyl and aryl are each optionally substituted;
R₁₁, R₁₂ and R₁₃ are each independently selected from the group consisting of hydrogen, alkyl, alkenyl, alkynyl, hydroxyl, hydroxyalkyl, halogen, amine, —NHC(NH)NH₂, —NHC(O)NH₂, —NHC(O)CH₃, —NHSO₂NH₂, —NHSO₂CH₃, —NHSO₂C₆H₅, —NHCHO wherein said alkyl, alkenyl, alkynyl and aryl are each optionally substituted;

R₁₄ is selected from the group consisting of imidazole, pyrazole, triazole, oxazole, isoxazole, thiazole, isothiazole, oxadiazole, thiadiazole and tetrazole, or substituted thereof, wherein each member of the group is optionally substituted;

R₁₅ is selected from the group consisting of indole, benzothiophene, benzoxazole, benzofuran, benzothiazole, benzimidazole, benzoxadiazole, benzothiadiazole, benzotriazole, pyrazolopyridine, imidazopyridine, pyrrolopyridine, pyrrolopyrimidine, indolizine, and purine, or substituted thereof, wherein each member of the group is optionally substituted;

L₁ to L₄ are each independently a bond or —(CH₂)ₙ—, wherein n is an integer between 0 and 10; and

Z₁ to Z₁₂ are each independently selected from the group consisting of —C(O)—, —CH₂—, —C(OH)—, —C(O)O-alkyl, and —C((O)alkyl)-.

The compounds represented by Formula I include the compound represented by the following Formula II:

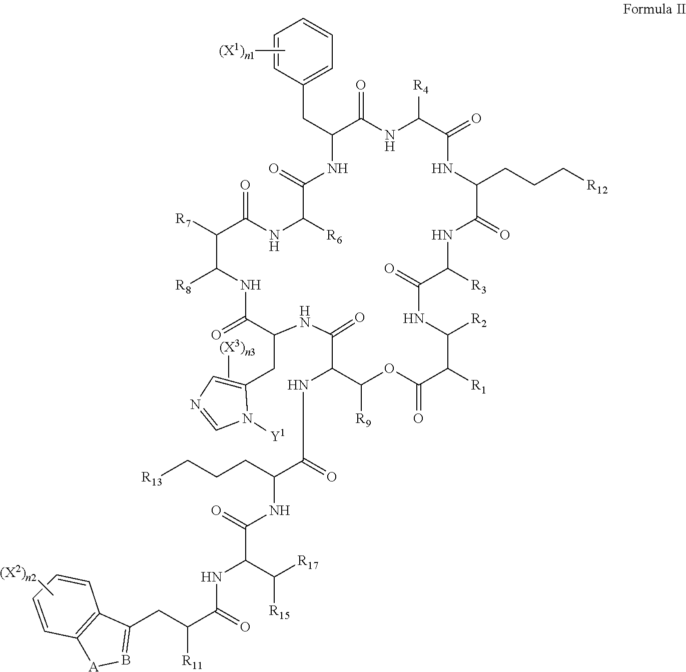

including stereochemically isomeric forms thereof:
wherein R₁ to R₄ and R₆ to R₁₃ are as defined above,
X¹ to X³ are each independently selected from the group consisting of halogen, hydroxyl, cyano, isocyano, nitro, amino, sulfanyl, carboxyaldehyde, hydroxycarbonyl, alkyl, haloalkyl, cyanoalkyl, and alkyloxy;
n1 to n3 are each independently an integer of 0 to 2;
Y₁ is selected from the group consisting of halogen, cyano, nitro, alkyl, alkoxy, alkylsulfanyl, alkyl substituted by halogen, —C(O)-alkyl, —C(O)—O-alkyl, and —NH—C(O)—O— alkyl;
A and B are each independently N or CR₁₈, wherein R₁₈ is selected from the group consisting of hydrogen, optionally substituted alkyl, optionally substituted alkenyl, optionally substituted alkynyl, and optionally substituted cycloalkyl.

The compounds represented by Formula I include the compound represented by the following Formula III:

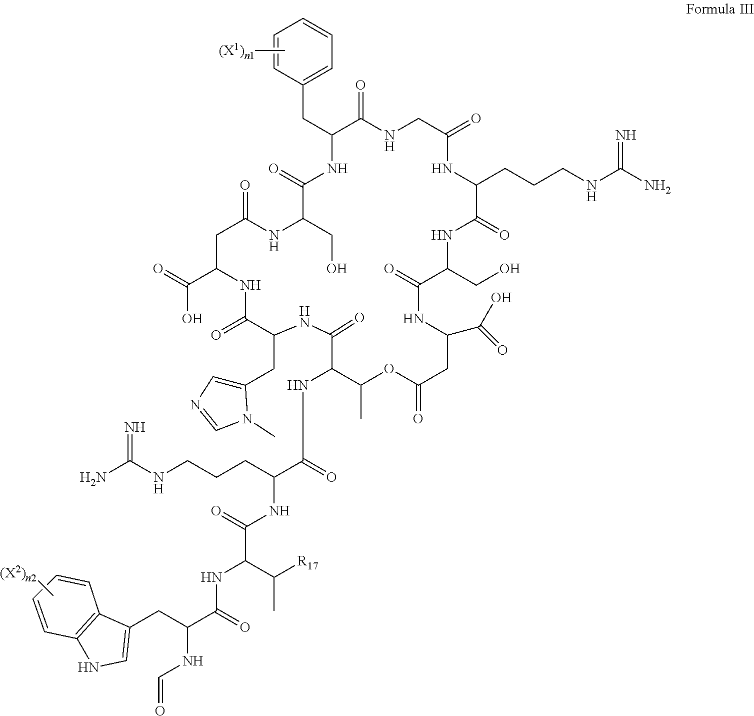

including a pharmaceutically acceptable salt, solvate or stereoisomer thereof.

The compounds represented by Formula I include the compound represented by the following Formula IV:

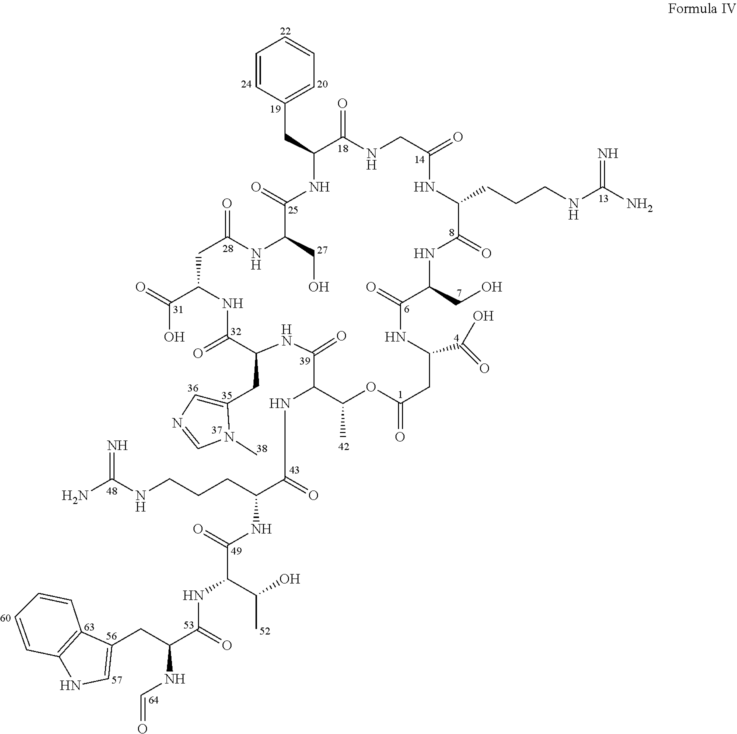 [SMILES: C[C@H]1OC(=O)C[C@@H](C(=O)O)NC(=O)[C@H](CO)NC(=O)[C@@H](CCCNC(=N)N)NC(=O)CNC(=O)[C@H](Cc2ccccc2)NC(=O)[C@@H](CO)NC(=O)C[C@@H](C(=O)O)NC(=O)[C@H](Cc2cncn2C)NC(=O)C1NC(=O)[C@@H](CCCNC(=N)N)NC(=O)[C@@H](NC(=O)[C@H](Cc1c[nH]c2ccccc12)NC=O)[C@@H](C)O]

including a pharmaceutically acceptable salt, solvate or stereoisomer thereof.

The present invention also relates to pharmaceutical compositions for treating a bacterial infection in a subject, particularly an M. tuberculosis infection. The compounds of Formulae I-IV, pharmaceutically acceptable salts, solvate or stereoisomer thereof can be useful, for example, for inhibiting the growth of M. tuberculosis, and/or for treating or preventing tuberculosis in patient.

The present invention is also directed to a method of treating tuberculosis in a subject in need of treatment thereof, comprising administering to the subject an effective amount of the compounds of Formulae I-IV, and uses of the compounds of Formulae I-IV for the treatment of tuberculosis.

The present invention is also directed to a composition for combatting, controlling or inhibiting a pest, comprising a pesticidally effective amount of the compounds of Formulae I-IV, pharmaceutically acceptable salts, solvate or stereoisomer thereof.

The present invention is also directed to a method of combatting, controlling or inhibiting a pest comprising exposing the pest to a pesticidally effective amount of the compounds of Formula I-IV or a salt, solvate or stereoisomer thereof.

Embodiments are either described in or will be apparent from the ensuing description, examples and appended claims.

### BRIEF DESCRIPTION OF THE DRAWINGS

FIG. 1 illustrates HPLC chromatogram and high-resolution ESI mass (HR-ESIMS) spectra of the compound of Formula IV. The compound of Formula IV shows peaks at m/z 1489.6764, 744.7402, and 496.8958 which correspond to the [M+H]⁺, [M+2H]²⁺, and [M+3H]³⁺ ions, respectively.

FIG. 2A illustrates the structure of the compound of Formula IV, and FIG. 2B illustrates the predicted biosynthetic gene cluster of the compound of Formula IV.

FIGS. 3A to 3F illustrate NMR spectra (700/175 Hz) of the compound of Formula IV in dimethyl sulfoxide (DMSO)-d6 (FIG. 3A, 1H; FIG. 3B, 13C; FIG. 3C, COSY; FIG. 3D, ROESY; FIG. 3E, 1H-13C HSQC; and FIG. 3F, 1H-13C HMBC).

FIGS. 4A to 4F illustrate NMR spectra for determination of the structure of the compound of Formula IV with dimethyl sulfoxide (DMSO) (FIG. 4A, 1H; FIG. 4B, 13C; FIG. 4C, COSY; FIG. 4D, ROESY; FIG. 4E, 1H-13C HSQC; and FIG. 4F, 1H-13C HMBC).

FIG. 5 illustrates 2D NMR key correlations for structural assignment of the compound of Formula IV. All correlations were measured in DMSO-d6 except for the HMBC correlation from H-41 to C-2 was recorded in D₂O.

FIG. 6 is BGC and proposed biosynthetic pathway of the compound of Formula IV, showing gene alignment of the BGC of the compound of Formula IV in the producer strain (A-E are NRPS genes and T1 and T2 are transporter genes) and the proposed biosynthetic pathway with 12 linear NRPS modules coded in genes A-E.

FIG. 7 illustrates a 1,1-ADEQUATE (600/150 MHz) NMR spectrum of the compound of Formula IV in DMSO-d6 supporting two β-aspartic acid moieties. The β protons in both moieties have cross-peaks with their respective carbonyl carbons at the γ positions.

FIG. 8 illustrates a ROESY (700 MHZ) NMR spectrum of the compound of Formula IV in 4% D₂O in H₂O supporting the β-aspartic acid linkage between a serine moiety and a methylated histidine moiety.

FIG. 9A to FIG. 9E show the efficacy of the compound of Formula IV. FIG. 9A shows the results of infecting mice by E. coli ATCC25922 via intraperitoneal injection, with antibiotics were administrated 1 h later. Survival ratios were monitored for 5 days. The experiment was repeated three times (n=4), with lines indicating the mean of experiments. Gentamicin (Gen) was used as a positive control. All treatment units are mg kg⁻¹. FIG. 9B shows optical microscopy and analysis of M. tuberculosis grown in 10×MIC of the compound of Formula IV. FIG. 9C illustrates cell elongation in the presence of antibiotics. FIGS. 9D and 9E show the time-dependent killing of early exponential (D) and stationary (E) cells of M. tuberculosis by 10×MIC the compound of Formula IV with n=3 biologically independent samples. Data are mean±s.d. c.f.u., colony-forming units. Significance was determined by one-way ANOVA with Tukey's post test. P<0.05*; P<0.0001,****.

FIG. 10 illustrates BacA homologs distributed among bacteria. A BacA homolog phylogenic tree was generated by using the maximum likelihood method based on the JTT matrix-based model 31. The tree with the highest log likelihood (−8349.22) is shown. Initial tree(s) for heuristic search were obtained automatically by applying Neighbor-Join and BioNJ algorithms to a matrix of pairwise distances estimated using a JTT model, and then selecting the topology with superior log likelihood value. The tree is drawn to scale, with branch lengths measured in the number of substitutions per site. The analysis involved 17 amino acid sequences. All positions containing gaps and missing data were eliminated. There were a total of 274 positions in the final dataset. Evolutionary analyses were conducted in MEGA7 (S. Kumar, et al., MEGA7: Molecular Evolutionary Genetics Analysis Version 7.0 for Bigger Datasets. Mol Biol Evol. 33, 1870-1874 (2016)). Protein sequences were obtained from previous study (D. J. Slotboom, et al., C. Bacterial multi-solute transporters. FEBS Lett 594, 3898-3907 (2020)).

FIG. 11A to FIG. 11D illustrate that the compound of Formula IV is transported into the cell via the ABC transporter BacA, targeting DNA gyrase A. FIG. 11A shows the frequency of generating drug-resistant mutants in M. tuberculosis. FIG. 11B illustrates resistant mutations to the compound of Formula IV mapped in BacA. FIG. 11C shows resistant mutations to the compound of Formula IV mapped onto the structure of the MtbGyrase DNA cleavage core. FIG. 11D depicts incorporation of the compound of Formula IV and resistance mechanism, where EVY indicates the compound of Formula IV, IM indicates the inner membrane, PG indicates peptidoglycan, and OM indicates the outer membrane.

FIG. 12 illustrates a graph showing an effect of the compound of Formula IV on macromolecular biosyntheses in E. coli WO153. Incorporation of ¹⁴C-thymidine (DNA), ¹⁴C-uridine (RNA), ¹⁴C-L-amino acid mixture (protein), ¹⁴C-acetic acid (fatty acid) and ¹⁴C-acetyl-glucosamine (peptidoglycan) was determined in cells treated with 8×MIC of the compound of Formula IV (grey bars). Ciprofloxacin (8×MIC), rifampicin (8×MIC), chloramphenicol (8×MIC), triclosan (8×MIC) and fosfomycin (8×MIC) were used as controls (white bars). There were n=3 biologically independent samples.

FIGS. 13A to 13C illustrate a bacterial gyrase and TopoIV poison for the compound of Formula IV. FIG. 13A: Ethidium bromide stained, native agarose gel based supercoiling assays for M. tuberculosis gyrase (top panel), E. coli gyrase (middle panel) and supercoil relaxation assays for E. coli TopoIV (bottom panel). The amount of enzyme is indicated in nM (0-20 nM), as added to 6 nM plasmid DNA. Assays were conducted in the absence or presence of 100 μM the compound of Formula IV. Bands for linear, nicked and supercoiled DNA are labeled. FIG. 13B: Quantitation and IC50 determination for the compound of Formula IV and moxifloxacin stimulated DNA cleavage activity using 20 nM M. tuberculosis gyrase and 0 to 100 μM drug. Cleavage was monitored by an ethidium bromide containing agarose gel-based cleavage assay. FIG. 13C: ATPase rates for 250 nM mtb gyrase (−) drug, 100 μM the compound of Formula IV and 100 μM moxifloxacin and reported in molecules of ATP consumed per minute per enzyme.

FIG. 14A to FIG. 14C show a crystal structure of the compound of Formula IV bound to M. tuberculosis gyrase. FIG. 14A: Drug-bound structures of gyrase bound to the compound of Formula IV (left), moxifloxacin (middle), and thiophenes (right). GyrA/gyrB heterodimer subunits are described in XX and YY with DNA depicted in gray. The binding location of the drug is outlined with the drugs represented as transparent surfaces. FIG. 14B: Close-up view of the compound of Formula IV binding site. The compound of Formula IV is depicted in stick representation with a transparent surface overlay. Hydrogen bonds are represented as dotted black lines and residues forming the binding pocket of the compound of Formula IV are represented as sticks and labeled. Key amino acids that comprise the compound of Formula IV peptide macrocycle are labeled. The overlap of the compound of Formula IV binding site with the thiophenes is indicated with an outline of the thiophene binding site. FIG. 14C: Intercalation of GyrB R482 at the site of DNA cleavage is shown for the compound of Formula IV bound M. tuberculosis gyrase structure and for the intercalation of the equivalent R458 in the thiophene bound S. aureus structure. The moxifloxacin binding site is illustrated as an outline.

FIGS. 15A and 15B show an electron density for the compound of Formula IV and the intercalating arginine. FIG. 15A: Electron density omit maps for the compound of Formula IV contoured at 1σ. Gyrase is depicted as a cartoon and the compound of Formula IV illustrated as sticks. FIG. 15B: Electron density omit maps for the compound of Formula IV bound M. tuberculosis gyrase structure (left) and the thiophene bound S. aureus gyrase structure (right).

FIG. 16 shows the compound of Formula IV and moxifloxacin stimulated cleavage activity of M. tuberculosis gyrase mutants.

FIG. 17 shows how mutations at the compound of Formula IV binding site affect the compound of Formula IV and moxifloxacin-induced cleavage. Plots represent quantitation of the compound of Formula IV and moxifloxacin induced cleavage in the presence of ATP. Fraction of linearized plasmid plotted at indicated concentrations of the compound of Formula IV or moxifloxacin (0-500 μM). Cleavage was conducted with 20 nM wild-type MtbGyrase or MtbGyrase GyrA mutants. Lines represent non-linear fits to the data, as in FIG. 13. Representative ethidium bromide containing agarose gel-based cleavage assays shown below. Nicked linear and uncleaved plasmid are indicated.

FIG. 18 shows supercoiling activities of M. tuberculosis gyrase mutants. Native agarose gel-based supercoiling activity assays using indicated amounts of M. tuberculosis gyrase (0-20 nM). Relaxed starting material and supercoiled product are indicated.

FIG. 19 shows that the compound of Formula IV binding pocket is concealed in the M. tuberculosis gyrase “ATPase open” state. The structure of S. cerevisiae TOP2 bound to DNA and nonhydrolyzable ATP analog illustrates the “ATPase closed” conformation of Type-II topoisomerases (left, PDBID: 4GFH). The structure of M. tuberculosis gyrase in the “ATPase open” state (right, PDBID: 6GAV) is shown. The inset shows the loop within the GyrB ATPase domains that is specific to M. tuberculosis family gyrases and how this loop occludes the compound of Formula IV binding site in the “ATPase open” conformation of the enzyme.

### DETAILED DESCRIPTION OF THE INVENTION

In vitro testing of the compound of Formula IV revealed the compounds and its derivatives have excellent potency in inhibiting the growth of M. tuberculosis. The compounds of Formulae I to IV and their pharmaceutically acceptable salts are expected to be useful for the treatment of M. tuberculosis.

In one aspect, the present invention is directed to novel macrocyclic depsipeptide compounds which have antibacterial activity that selectively kill M. tuberculosis. The compounds and their derivatives, and their pharmaceutically acceptable salts, can be useful, for example, for the treatment of bacterial infections, for example, mycobacterial infections. More particularly, the present invention includes the compounds represented by the following Formula I, a pharmaceutically acceptable salt, solvate or stereoisomer thereof:

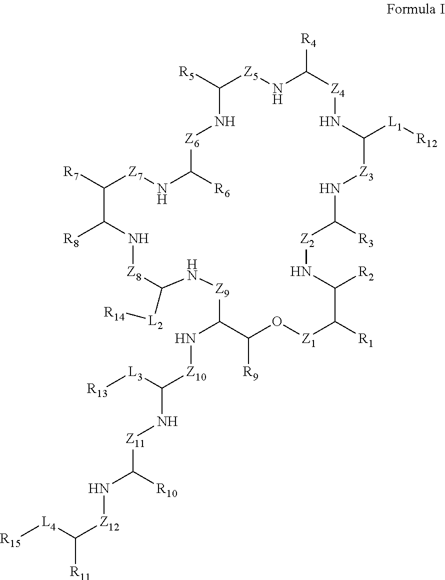

including a pharmaceutically acceptable salt, solvate or stereoisomer thereof:
wherein, in Formula I,
R₁ to R₁₀ are each independently selected from the group consisting of hydrogen, alkyl, alkenyl, alkynyl, hydroxyl, hydroxyalkyl, halogen, —CN, —O-alkyl, —C(O)-alkyl, —C(O)O-alkyl, —C(O)OH, —C(O)NH₂, —C(O)NH-alkyl, —NH₂, —NO₂, —CF₃, —NH-alkyl, —N— (alkyl)₂, —NHC(O)-alkyl, -arylalkyl, -alkylaryl, alkylheteroaryl, wherein said alkyl, alkenyl, alkynyl and aryl are each optionally substituted;
R₁₁, R₁₂ and R₁₃ are each independently selected from the group consisting of hydrogen, alkyl, alkenyl, alkynyl, hydroxyl, hydroxyalkyl, halogen, amine, —NHC(NH)NH₂, —NHC(O)NH₂, —NHC(O)CH₃, —NHSO₂NH₂, —NHSO₂CH₃, —NHSO₂C₆H₅, —NHCHO wherein said alkyl, alkenyl, alkynyl and aryl are each optionally substituted;
R₁₄ is selected from the group consisting of imidazole, pyrazole, triazole, oxazole, isoxazole, thiazole, isothiazole, oxadiazole, thiadiazole and tetrazole, wherein each member of the group is optionally substituted;
R₁₅ is selected from the group consisting of indole, benzothiophene, benzoxazole, benzofuran, benzothiazole, benzimidazole, benzoxadiazole, benzothiadiazole, benzotriazole, pyrazolopyridine, imidazopyridine, pyrrolopyridine, pyrrolopyrimidine, indolizine, and purine, wherein each member of the group is optionally substituted;
L₁ to L₄ are each independently a bond or —(CH₂)ₙ—, wherein n is an integer between 0 and 10; and
Z₁ to Z₁₂ are each independently selected from the group consisting of —C(O)—, —CH₂—, —C(OH)—, —C(O)O-alkyl, and —C((O)alkyl)-.

In some embodiments, the compounds represented by Formula I may include a compound represented by the following Formula II:

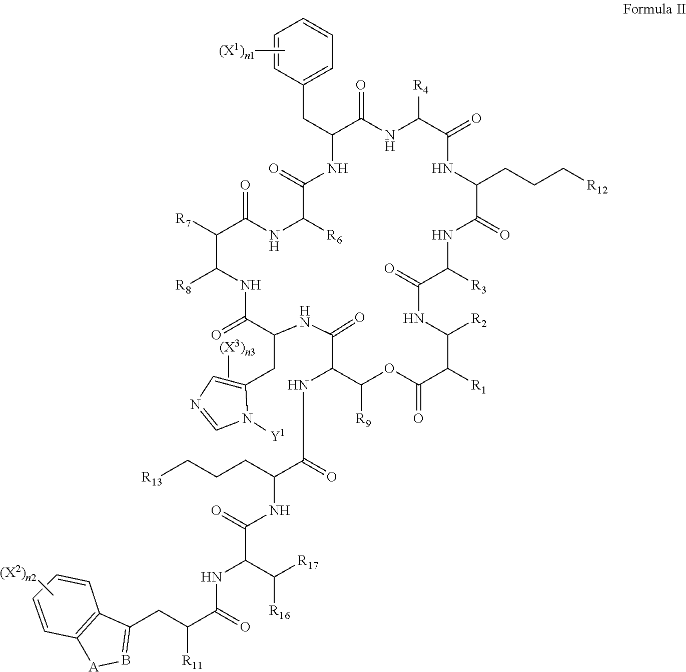

including a pharmaceutically acceptable salt, solvate or stereoisomer thereof:
wherein R₁ to R₄ and R₆ to R₁₃ are as defined above;
R₁₆ and R₁₇ are each independently selected from the group consisting of hydrogen, alkyl, alkenyl, alkynyl, hydroxyl, hydroxyalkyl, halogen, —CN, —O-alkyl, —C(O)-alkyl, —C(O)O-alkyl, —C(O)OH, —C(O)NH₂, —C(O)NH-alkyl, —NH₂, —NO₂, —CF₃, —NH-alkyl, —N— (alkyl)₂, —NHC(O)-alkyl, -aryl, -alkylaryl, alkylheteroaryl, wherein said alkyl, alkenyl, alkynyl and aryl are each optionally substituted;
X¹ to X³ are each independently selected from the group consisting of halogen, hydroxyl, cyano, isocyano, nitro, amino, sulfanyl, carboxyaldehyde, hydroxycarbonyl, alkyl, haloalkyl, cyanoalkyl, and alkyloxy;
n1 to n3 are each independently an integer of 0 to 2;
Y₁ is selected from the group consisting of halogen, cyano, nitro, alkyl, alkoxy, alkylsulfanyl, alkyl substituted by halogen, —C(O)-alkyl, —C(O)—O-alkyl, and —NH—C(O)—O— alkyl;
A and B are each independently N or CR₁₈, wherein R₁₈ is selected from the group consisting of hydrogen, optionally substituted alkyl, optionally substituted alkenyl, optionally substituted alkynyl, and optionally substituted cycloalkyl.

In some embodiments, in Formulae I and II, R₁, R₄ and R₇ may be hydrogen.

In some embodiments, in Formulae I and II, R₂ and R₈ may be —C(O)OH. In some embodiments, in Formulae I and II, R₃ and R₆ may be —CH₂OH. In some embodiments, in Formulae I and II, R₉ may be —CH₃. In some embodiments, in Formulae I and II, R₁₀ may be —CH(OH)CH₃. In some embodiments, in Formulae I and II, R₁ may be —NHCHO. In some embodiments, in Formulae I and II, R₁₂ and R₁₃ may be —NHC(NH)NH₂.

In some embodiments, the compounds represented by Formula I may include a compound represented by Formula III:

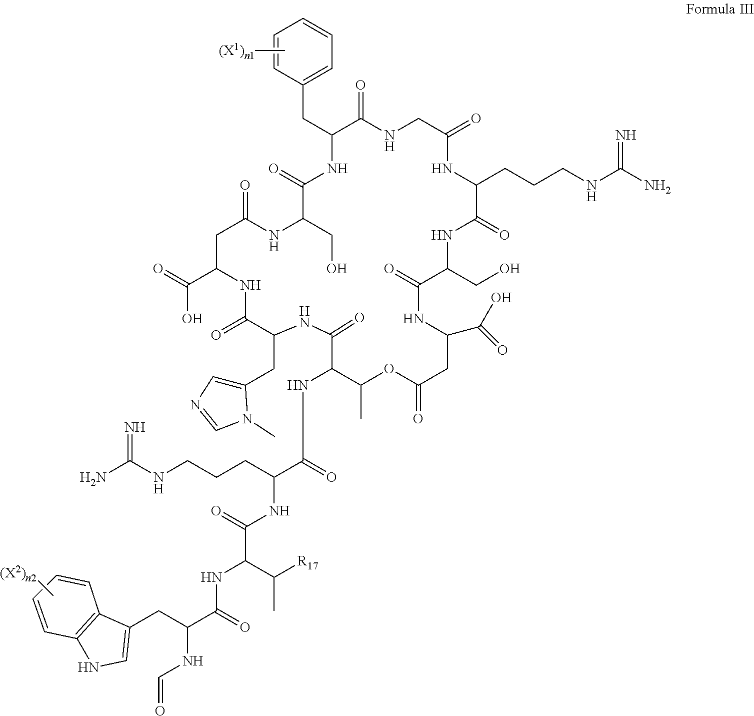

including a pharmaceutically acceptable salt, solvate or stereoisomer thereof:
wherein R₁₇ and X¹ and X² are as defined above.

In some embodiments, the compounds represented by Formula I may include a compound represented by Formula III(a):

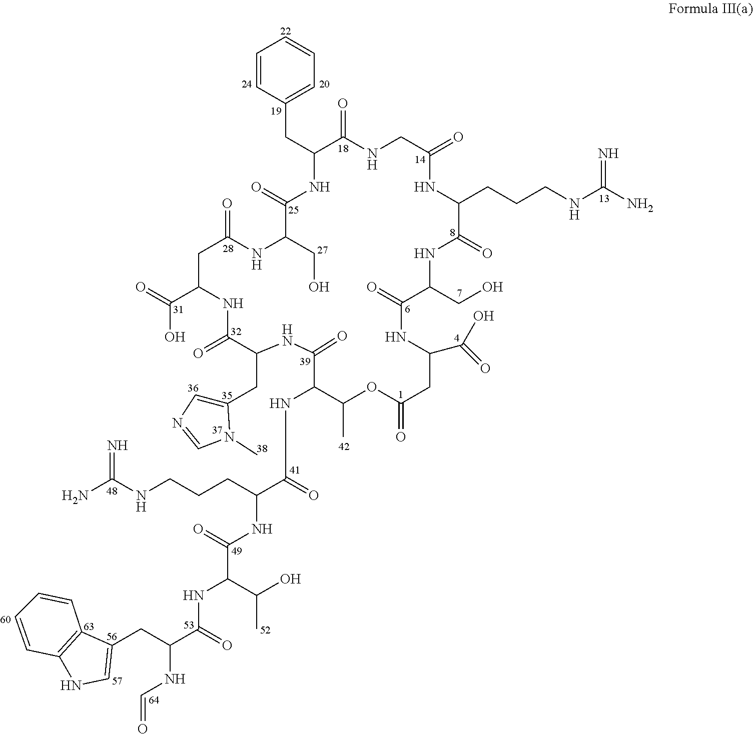 [SMILES: CC(O)C(NC(=O)C(Cc1c[nH]c2ccccc12)NC=O)C(=O)NC(CCCNC(=N)N)C(=O)NC1C(=O)NC(Cc2cncn2C)C(=O)NC(C(=O)O)CC(=O)NC(CO)C(=O)NC(Cc2ccccc2)C(=O)NCC(=O)NC(CCCNC(=N)N)C(=O)NC(CO)C(=O)NC(C(=O)O)CC(=O)OC1C]

including stereochemically isomeric forms thereof.

In some embodiments, the compounds represented by Formula I may include a compound represented by Formula IV:

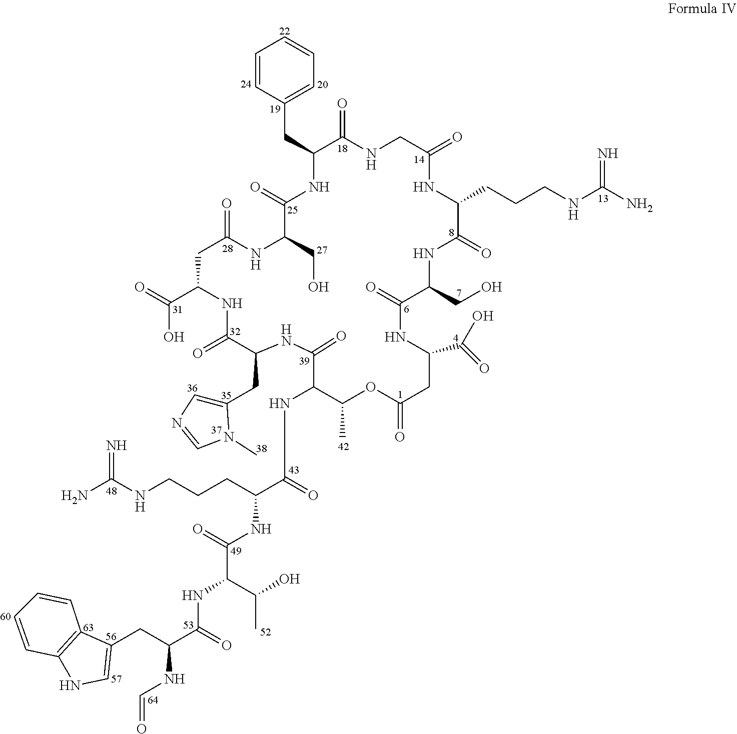 [SMILES: C[C@H]1OC(=O)C[C@@H](C(=O)O)NC(=O)[C@H](CO)NC(=O)[C@@H](CCCNC(=N)N)NC(=O)CNC(=O)[C@H](Cc2ccccc2)NC(=O)[C@@H](CO)NC(=O)C[C@@H](C(=O)O)NC(=O)[C@H](Cc2cncn2C)NC(=O)C1NC(=O)[C@@H](CCCNC(=N)N)NC(=O)[C@@H](NC(=O)[C@H](Cc1c[nH]c2ccccc12)NC=O)[C@@H](C)O]

including stereochemically isomeric forms thereof.

In one aspect, the present invention is directed to one or more stereochemically pure represented by Formulae I to IV. In another aspect, the present invention is directed to a stereochemically pure compound represented by Formula IV isolated and purified according to standard techniques well known to the person skill in the art and examples of such methods include chromatographic techniques such as column chromatography and HPLC. One technique of particular usefulness in purifying the compounds is preparative liquid chromatography using mass spectrometry as a means of detecting the purified compounds emerging from the chromatography column.

In one aspect, the present invention is directed to a pharmaceutical composition for treating an infection caused by mycobacterium in a subject comprising a therapeutically effective amount of one of the compounds represented by Formulae I to IV or a pharmaceutically acceptable salt, solvate or stereoisomer thereof. In some embodiments, the pharmaceutical composition may further comprise at least one pharmaceutically acceptable carrier, excipient or diluent. In some embodiments, the pharmaceutical composition may be in a form of topical administration, systemic administration, parenteral administration, subcutaneous administration, or transdermal administration, rectal administration, oral administration, intravaginal administration, intranasal administration, intrabronchial administration, intraocular administration, intra-aural administration, intravenous administration, intramuscular administration, or intraperitoneal administration. In some embodiments, the pharmaceutical composition may further comprise at least one additional therapeutic agent.

In some embodiments, the pharmaceutical composition may be obtained by culturing a microorganism having an ability to produce the compound in a nutrient medium. In some embodiments, the pharmaceutical composition may be obtained by culturing Photorhabdus noenieputensis DSM 25462.

In one aspect, the present invention is directed to a method of treating a disease or an infection caused by a bacterium in a subject in need thereof, comprising administering a therapeutically effective amount of one or more of the compounds represented by Formulae I to IV, or pharmaceutically acceptable salts thereof, solvate or stereoisomer thereof. In some embodiments, the compounds represented by Formulae I to IV or pharmaceutically acceptable salts thereof, solvate or stereoisomer thereof may be administered in combination with a pharmaceutically acceptable carrier to form a pharmaceutical composition. In some embodiments, the infection may be a respiratory infection, a skin or skin structure infection, a urinary infection, an intra-abdominal infection, a blood stream infection, or a gastrointestinal infection. In some embodiments, the infection may be a Mycobacterium tuberculosis infection. In some embodiments, the bacterium may be a Gram-positive bacterium. In some embodiments, the Gram-positive bacterium may be selected from the group consisting of Streptococcus, Staphylococcus, Enterococcus, Corynebacteria, Listeria, Bacillus, Erysipelothrix, Mycobacterium, Clostridium, and Actinomycetales.

In some embodiments, the Gram-positive bacterium may be elected from the group consisting of Staphylococcus aureus, Staphylococcus epidermidis, Staphylococcus haemolyticus, Staphylococcus hominis, Staphylococcus saprophyticus, Staphylococcus aureus, Streptococcus pneumoniae, Streptococcus pyogenes, Streptococcus agalactiae, Streptococcus dysgalactiae, Streptococcus avium, Streptococcus bovis, Streptococcus lactis, Streptococcus sangius, Streptococcus anginosus, Streptococcus intermedius, Streptococcus constellatus, Viridans streptococci, Enterococcus faecalis, Enterococcus faecium, Clostridium difficile, Clostridium clostridiiforme, Clostridium innocuum, Clostridium perfringens, Clostridium tetani, Mycobacterium tuberculosis, Mycobacterium avium, Mycobacterium intracellulare. Mycobacterium kansasii, Mycobacterium gordonae, Mycobacteria sporozoites, Listeria monocytogenes, Bacillus subtilis, Bacillus anthracis, Corynebacterium diphtherias, Corynebacterium jeikeium, Corynebacterium sporozoites, Erysipelothrix rhusiopathiae, and Actinomyces israelii.

In some embodiments, the bacterial infection may be a respiratory infection, a skin or skin structure infection, urinary infection, an intra-abdominal infection, a blood stream infection, or a gastrointestinal infection.

In some embodiments, the infection may be caused by Mycobacterium africanum, Mycobacterium avium. Mycobacterium bovis, Mycobacterium canetti, Mycobacterium caprae, Mycobacterium colombiense, Mycobacterium avium hominissuis, Mycobacterium intracellulare, Mycobacterium microti, Mycobacterium mungi, Mycobacterium orygis, Mycobacterium pinnipedii, Mycobacterium avium silvaticum, Mycobacterium suricattae, or Mycobacterium tuberculosis, Mycobacterium ulcerans. Mycobacterium xenopi.

In some embodiments, the compounds represented by Formulae I to IV may be administered in combination or alternation with an additional therapeutic agent selected from acedapsone, clofazimine, dapsone, desoxyfructo-serotonin, ethambutol, ethionamide, isoniazid, moxifloxacinor, pyrazinamide, rifapentine, streptomycin, sulfameter, thiacetazone, thalidomide, combinations thereof.

In some embodiments, the subject may be a mammal. In some embodiments, the subject may be a human. In some embodiments, the subject may be a nonhuman.

In some embodiments, the administering step may be topical administration, systemic administration, parenteral administration, subcutaneous administration, or transdermal administration, rectal administration, oral administration, intravaginal administration, intranasal administration, intrabronchial administration, intraocular administration, intra-aural administration, intravenous administration, intramuscular administration, or intraperitoneal administration.

In one aspect, the present invention is directed to a method for alleviating a symptom associated with tuberculosis, comprising administering to a subject in need thereof an effective amount of at least one of the compounds represented by Formulae I to IV.

In another aspect, the present invention is directed to a method of inhibiting and/or controlling pests, comprising delivering to the pests a pesticidally effect amount of at least one of the compounds represented by Formula I to IV. In some embodiments, the pests may be insect pests or parasitic pests. In some embodiments, the parasitic pest may be an insect pest of the order Acarina or nematodes. In some embodiments, the parasitic pest may be animal parasitic nematodes. In some embodiments, the parasitic pest may be nematodes of the order Spirurida. In some embodiments, the parasitic pest may be heartworm.

Embodiments of the present invention relate to an antibiotic that selectively kills M. tuberculosis. A novel cyclic depsipeptide DNA gyrase inhibitor, a compound of Formula IV, was isolated from culture extract of Photorhabdus noenieputensis (P. noenieputensis) DSM 25462 and shows potent activity against M. tuberculosis and low activity against other pathogens. It demonstrates no cytotoxicity against human cell lines. The compound of Formula IV is smuggled into the cell through BacA, a multi-solute transporter for hydrophilic molecules and targets DNA gyrase subunit A, which explains the mechanism of its selectivity. Surprisingly, the compound of Formula IV acts at a site known to be targeted by thiopene agents, an allosterically acting class of gyrase antagonists, distinguishing its mode of action from widely-used fluoroquinolone antibiotics.

This application also relates to a method of combatting, controlling or inhibiting a pest comprising exposing a pest to a pesticidally effective amount of a compound described herein or a salt, hydrate or prodrug thereof.

As used herein, “pesticidally effect amount” refers to an amount of compound described herein that is able to bring about death to at least one pest, or noticeably reduce pest growth, feeding, or normal physiological development. This amount will vary depending on such factors as, for example, the specific target pests to be controlled, the specific environment, location, plant, crop, or agricultural site to be treated, the environmental conditions, and the method, rate, concentration, stability, and quantity of application of the pesticidally-effective compound. As used herein, “pest” includes, but is not limited to insects, fungi, bacteria, nematodes, mites, ticks and the like.

As used herein and unless otherwise indicated, the term “compounds of the invention” means, collectively, the compounds of Formulae I to IV and pharmaceutically acceptable salts, solvate or stereoisomer thereof as well as specific compounds depicted herein. The compounds of the invention are identified herein by their chemical structure and/or chemical name. Where a compound is referred to by both a chemical structure and a chemical name, and that chemical structure and chemical name conflict, the chemical structure is determinative of the compound's identity. The compounds of the invention may contain one or more chiral centers and/or double bonds and, therefore, exist as stereoisomers, such as double-bond isomers (i.e., geometric isomers), enantiomers, or diastereomers. According to the invention, the chemical structures depicted herein, and therefore the compounds of the invention, encompass all of the corresponding compound's enantiomers and stereoisomers, that is, both the stereomerically pure form (e g., geometrically pure, enantiomerically pure, or diastereomerically pure) and enantiomeric and stereoisomeric mixtures. Enantiomeric and stereoisomeric mixtures can be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically- or enantiomerically-pure intermediates, reagents, and catalysts by well-known asymmetric synthetic methods.

As used herein and unless otherwise indicated, the term “stereochemically pure” means a composition that comprises one stereoisomer of the compound and is substantially free of other stereoisomers of that compound. In some embodiments, stereochemically pure composition of comprises a compound which has 80% or greater by weight of the indicated stereoisomer and 20% or less by weight of other stereoisomers. In some embodiments, the compounds of Formulae I to IV have 80%, 85%, 90%, 95%, 98% or 99% or greater by weight of the stated stereo isomer and 20%, 15%, 10%, 5%, 2%, or 1% or less by weight of other stereoisomers.

As used herein and unless otherwise indicated, the term “alkyl” means a substituted or unsubstituted, saturated, linear or branched hydrocarbon chain radical. Examples of alkyl groups include, but are not limited to, C1-C15 linear, branched or cyclic alkyl, such as methyl, ethyl, propyl, isopropyl, cyclopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, sec-butyl, t-butyl, cyclobutyl, pentyl, isopentyl, neopentyl, hexyl, and cyclohexyl and longer alkyl groups, such as heptyl, octyl, nonyl and decyl. An alkyl can be unsubstituted or substituted with one or two suitable substituents.

As used herein and unless otherwise indicated, the terms “alkoxy” or “alkyloxy” means an —O-alkyl, wherein alkyl is as defined herein. An alkoxy may be unsubstituted or substituted with one or two suitable substituents. Preferably, the alkyl chain of an alkyloxy is from 1 to 5 carbon atoms in length, referred to herein, for example, as “C1-C5 alkoxy.” In one embodiment, the alkyl chain of an alkyloxy is from 1 to 10 carbon atoms in length, referred to herein, for example, as “C1-C10 alkoxy.”

As used herein and unless otherwise indicated, the terms “alkene” or “alkenyl group” means a monovalent linear, branched or cyclic hydrocarbon chain having one or more double bonds therein. The double bond of an alkene can be unconjugated or conjugated to another unsaturated group. An alkene can be unsubstituted or substituted with one or two suitable substituents. Suitable alkenes include, but are not limited to C2-C8 alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkene can be unsubstituted or substituted with one or two suitable substituents.

As used herein and unless otherwise indicated, the terms “alkynyl” means an unsaturated straight or branched hydrocarbon having at least one carbon-carbon triple bond. Examples of alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl.

As used herein and unless otherwise indicated, the term “hydroxyl” is represented by the formula —OH.

As used herein and unless otherwise indicated, the term “alkylaryl” means alkyl groups one or more of hydrogen is substituted with an aryl group, including -alkylaryl structure which is attached to the parent molecule via the alkyl group and -arylalkyl structure which is attached to the parent molecule via the aryl group. Examples of alkylaryl groups include, but are not limited to, benzyl, phenethyl, benzyl(phenylmethyl), and naphthylmethyl. The alkylaryl group may be substituted or unsubstituted.

As used herein and unless otherwise indicated, the term “alkylheteroaryl” means alkyl groups wherein one or more of hydrogen is substituted with a heteroaryl group, including -alkylheteroaryl structure which is attached to an adjacent structure via the alkyl group and -heteroarylalkyl structure which is attached to an adjacent structure via the heteroaryl group.

As used herein and unless otherwise indicated, the term “amine” means NR^{a}R^{b} groups, wherein R^{a} and R^{b} are independently hydrogen, or substituted or unsubstituted alkyl, alkenyl, alkynyl, cycloalkyl, aryl, aralkyl, heterocycloalkyl, or heterocyclyl group. In some embodiments, amine includes alkylamino, dialkylamino, arylamino, and alkylarylamino. Examples of amine includes NH₂, methylamino, dimethylamino, ethylamino, diethylamino, propylamino, isopropylamino, phenylamino, and benzylamino.

As used herein and unless otherwise indicated, the term “alkylsulfanyl” means an alkyl group bonded to the parent molecule via a sulfur atom.

As used herein and unless otherwise indicated, the term “hydroxyalkyl” means that at least one hydroxy group, as defined herein, is added to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

As used herein and unless otherwise indicated, the term “aryl” or “aromatic ring” means a monocyclic or polycyclic conjugated ring structure that is well known in the art. Examples of suitable aryl groups or aromatic rings include, but are not limited to, phenyl, tolyl, anthacenyl, fluorenyl, indenyl, azulenyl, and naphthyl. An aryl group can be unsubstituted or substituted with one or two suitable substituents. In one embodiment, the aryl group is a monocyclic ring, wherein the ring comprises 6 carbon atoms, referred to herein as “C6 aryl.”

As used herein and unless otherwise indicated, the term “substituted aryl” includes an aryl group optionally substituted with one or more functional groups, such as halo, alkyl, haloalkyl (e g., trifluoromethyl), alkoxy, haloalkoxy (e.g., difluoromethoxy), alkenyl, alkynyl, aryl, heteroaryl, arylalkyl, aryloxy, aryloxyalkyl, arylalkoxy, alkoxy carbonyl, alkylcarbonyl, arylcarbonyl, arylalkenyl, aminocarbonylaryl, arylthio, arylsulfmyl, arylazo, heteroarylalkyl, heteroaryl alkenyl, heteroaryloxy, hydroxy, nitro, cyano, amino, substituted amino wherein the amino includes 1 or 2 substituents (which are optionally substituted alkyl, aryl or any of the other substituents recited herein), thiol, alkylthio, arylthio, heteroarylthio, arylthioalkyl, alkoxyarylthio, alkylaminocarbonyl, arylaminocarbonyl, aminocarbonyl, alkylcarbonyloxy, arylcarbonyloxy, alkylcarbonylamino, arylcarbonylamino, arylsulfmyl, arylsulfmylalkyl, arylsulfonylamino, or arylsulfonaminocarbonyl and/or any of the alkyl substituents recited herein.

As used herein and unless otherwise indicated, the term “heteroaryl” as used herein alone or as part of another group refers to a 5- to 7-membered aromatic ring which includes 1, 2, 3 or 4 hetero atoms such as nitrogen, oxygen or sulfur and such rings fused to an aryl, cycloalkyl, heteroaryl or heterocycloalkyl ring (e g. benzothiophenyl, indolyl), and includes possible N-oxides. “Substituted heteroaryl” includes a heteroaryl group optionally substituted with 1 to 4 substituents, such as the substituents included above in the definition of “substituted alkyl” and “substituted cycloalkyl.” Substituted heteroaryl also includes fused heteroaryl groups which include, for example, quinoline, isoquinoline, indole, isoindole, carbazole, acridine, benzimidazole, benzofuran, isobenzofuran, benzothiophene, phenanthroline, purine, and the like.

As used herein and unless otherwise indicated, the terms “heterocyclo,” “heterocycle,” or “heterocyclic ring,” as used herein, refer to an unsubstituted or substituted stable 5- to 7-membered monocyclic ring system which may be saturated or unsaturated, and which consists of carbon atoms and from one to four heteroatoms selected from N, O or S, and wherein the nitrogen and sulfur heteroatoms may optionally be oxidized, and the nitrogen heteroatom may optionally be quaternized. The heterocyclic ring may be attached at any heteroatom or carbon atom which results in the creation of a stable structure. Examples of such heterocyclic groups include, but are not limited to, piperidinyl, piperazinyl, oxopiperazinyl, oxopiperidinyl, oxopyrrolidinyl, oxoazepinyl, azepinyl, pyrrolyl, pyrrolidinyl, furanyl, thienyl, pyrazolyl, pyrazolidinyl, imidazolyl, imidazolinyl, imidazolidinyl, pyridyl, pyrazinyl, pyrimidinyl, pyridazinyl, oxazolyl, oxazolidinyl, isooxazolyl, isoxazolidinyl, morpholinyl, thiazolyl, thiazolidinyl, isothiazolyl, thiadiazolyl, tetrahydropyranyl, thiamorpholinyl, thiamorpholinylsulfoxide, thiamorpholinylsulfone, and oxadiazolyl.

As used herein and unless otherwise indicated, the term “substituted” or “optionally substituted” may indicate that a chemical moiety referred to, for example, alkyl, aryl, heteroaryl, may be unsubstituted or substituted with one or more groups including, without limitation, alkyl, alkenyl, alkynyl, cycloalkyl, arylalkyl, aryl, heterocycle, heteroaryl, hydroxyl, amino, alkoxy, halogen, carboxy, carbalkoxy, carboxamido, monoalkylaminosulfmyl, dialkylaminosulfmyl, monoalkylaminosulfonyl, dialkylaminosulfonyl, alkylsulfonylamino, hydroxysulfonyloxy, alkoxysulfonyloxy, alkylsulfonyloxy, hydroxy sulfonyl, alkoxysulfonyl, alkylsulfonylalkyl, monoalkylaminosulfonylalkyl, dialkylaminosulfonylalkyl, monoalkylaminosulfmylalkyl, dialkylaminosulfmylalkyl and the like. For example, optionally substituted alkyl may include both propyl and 2-chloro-propyl. Additionally, “optionally substituted” is also inclusive of embodiments where the named substituent or substituents have multiple substituents rather than simply a single substituent. For example, optionally substituted aryl may include both phenyl and 3-methyl-5-ethyl-6-chloro-phenyl.

As used herein and unless otherwise indicated, the term “cycloalkyl” includes saturated or partially unsaturated (containing 1 or more double bonds) cyclic hydrocarbon groups containing 1 to 3 rings, including monocyclicalkyl, bicyclicalkyl and tricyclicalkyl, containing a total of C3 to C20 carbons forming the rings, or about C3 to C10 carbons, forming the ring and which may be fused to 1 or 2 aromatic rings as described for aryl, which include cyclopropyl, cyclobutyl, cyclopentyl, cyclohexyl, cycloheptyl, cyclooctyl, cyclodecyl, cyclododecyl, and cyclohexenyl.

As used herein and unless otherwise indicated, the term “substituted cycloalkyl” includes a cycloalkyl group optionally substituted with 1 or more substituents such as halogen, alkyl, substituted alkyl, alkoxy, hydroxy, aryl, substituted aryl, aryloxy, cycloalkyl, alkylamido, alkanoylamino, oxo, acyl, arylcarbonylamino, amino, nitro, cyano, thiol and/or alkylthio and/or any of the substituents included in the definition of “substituted alkyl.”

As used herein and unless otherwise indicated, the term “cycloalkenyl” includes a nonaromatic monocyclic or bicyclic carbocylic ring containing at least one double bond. Examples of cycloalkenyl groups include, but are not limited to, cyclopropenyl, cyclobutenyl, cyclopentenyl, cyclohexenyl, cycloheptenyl, cyclooxtenyl and the like.

As used herein and unless otherwise indicated, the term “aryloxy” means an —O-aryl group, wherein aryl is as defined herein. An aryloxy group can be unsubstituted or substituted with one or two suitable substituents. Preferably, the aryl ring of an aryloxy group is a monocyclic ring, wherein the ring comprises C6 carbon atoms, referred to herein as “C6 aryloxy.”

As used herein and unless otherwise indicated, the term “ether” means a group of formula alkyl-O-alkyl, alkyl-O-alkynyl, alkyl-O-aryl, alkenyl-O-alkenyl, alkenyl-O-alkynyl, alkenyl-O-aryl, alkynyl-O-alkynyl, alkynyl-O-aryl, aryl-O-aryl, wherein “alkyl”, “alkenyl”, “alkynyl” and “aryl” are defined herein.

As used herein and unless otherwise indicated, the term “carboxy” means a radical of the formula:—COOH.

As used herein and unless otherwise indicated, the term “halogen” or “halo” means fluorine, chlorine, bromine or iodine.

As used herein and unless otherwise indicated, the phrase “pharmaceutically acceptable salt(s),” as used herein includes but is not limited to salts of acidic or basic groups that may be present in the compounds (including the compounds of the invention) used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions including, but not limited to, sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium and iron salts.

As used herein and unless otherwise indicated, the term “solvate” means forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

As used herein and unless otherwise indicated, the term “excipient” means a pharmaceutically acceptable, inactive substance used as a carrier for the pharmaceutically active ingredient(s) and includes antiadherents, binders, coatings, disintegrants, fillers, diluents, flavors, bulkants, colours, glidants, dispersing agents, wetting agents, lubricants, preservatives, sorbents and sweeteners.

As used herein and unless otherwise indicated, the term “administer” and “administration” can also include administering a combination of compounds. Thus, administration may be in the form of dosing an organism with a compound or combination of compounds, such that the organism's circulatory system will deliver a compound or combination of compounds to the target area, including but not limited to a cell or cells, synaptic junctions and circulation. Administration may also mean that a compound or combination of compounds is placed in direct contact with an organ, tissue, area, region, cell or group of cells, such as but not limited to direct injection of the combination of compounds.

In some embodiments, a combination of compounds can be administered, and thus the individual compounds can also be said to be co-administered with one another. As used herein, “co-administer” indicates that each of at least two compounds is administered during a time frame wherein the respective periods of biological activity or effects overlap. Thus, the term co administer includes sequential as well as coextensive administration of the individual compounds, at least one of which is a compound of the present invention. Accordingly, “administering” a combination of compounds according to some of the methods of the present invention includes sequential as well as coextensive administration of the individual compounds of the present invention. Likewise, the phrase “combination of compounds” indicates that the individual compounds are co-administered, and the phrase “combination of compounds” does not mean that the compounds must necessarily be administered contemporaneously or coextensively. In addition, the routes of administration of the individual compounds need not be the same.

As used herein and unless otherwise indicated, the terms “treat” and “treatment” refer to a slowing of or a reversal of the progress of the disease or infection. Treating a disease includes treating a symptom and/or reducing the symptoms of the disease or infection. The term “preventing” refers to a slowing of the disease or of the onset of the disease, infection or the symptoms thereof. Preventing a disease or infection can include stopping the onset of the disease, infection or symptom thereof.

As used herein and unless otherwise indicated, the term “subject” may be an animal, vertebrate animal, mammal, rodent (e.g., a guinea pig, a hamster, a rat, a mouse), a murine (e g., a mouse), a canine (e g., a dog), a feline (e.g. a cat), an equine (e.g., a horse), a primate, a simian (e.g., a monkey or ape), a monkey (e.g., marmoset, a baboon), an ape (e.g., gorilla, chimpanzee, orangutan, gibbon), or a human.

As used herein and unless otherwise indicated, the term “dosage unit” refers to a physically discrete unit, such as a capsule or tablet suitable as a unitary dosage for a subject. Each unit contains a predetermined quantity of at least one of the compounds of Formulae I to IV which was discovered or believed to produce the desired pharmacokinetic profile which yields the desired therapeutic effect. The dosage unit is composed of at least one compound of Formulae I to IV in association with at least one pharmaceutically acceptable carrier, salt, excipient or a combination thereof. The term “dose” or “dosage” refers to the amount of active ingredient that an individual takes or is administered at one time.

As used herein and unless otherwise indicated, the term “therapeutically effective amount” refers to the amount sufficient to produce a desired biological effect in a subject. Accordingly, a therapeutically effective amount of a compound may be an amount which is sufficient to treat or prevent a disease or infection, and/or delay the onset or progression of a disease or infection, and or alleviate one or more symptoms of the disease or infection, when administered to a subject suffered from or susceptible to that disease or infection. A “pharmaceutically acceptable carrier” or “pharmaceutically acceptable excipient” herein refers to a non-API (where API refers to Active Pharmaceutical Ingredient) substances such as disintegrators, binders, fillers, and lubricants used in formulating pharmaceutical products. They are generally safe for administering to humans.

As used herein and unless otherwise indicated, the term “pharmaceutically acceptable” means approved by a regulatory agency of the Federal or a state government or listed in the U.S. Pharmacopeia or other generally recognized pharmacopeia for use in animals, and more particularly in humans. The term “vehicle” refers to a diluent, adjuvant, excipient, or carrier with which a compound of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. In one embodiment, when administered to a patient, the combination of compounds of the invention and pharmaceutically acceptable vehicles are sterile. Water and/or oils are one vehicle when the combination of compounds of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present combination of compounds, if desired, can also contain minor amounts of wetting or emulsifying agents, or pH buffering agents.

As used herein and unless otherwise indicated, the term “pesticidally effective amount” means a quantity of a compound that has pesticidal activity when present in the environment of a pest. For each substance or organism, the pesticidally effective amount is determined empirically for each pest affected in a specific environment. In some embodiment, the pesticidally effective amount is an amount of the compound or composition needed to achieve an observable effect on growth, including the effects of necrosis, death, retardation, prevention, and removal, destruction, or otherwise diminishing the occurrence and activity of the target pest organism. The pesticidally effective amount can vary for the various mixtures/compositions used in the invention. A pesticidally effective amount of the mixtures/compositions will also vary according to the prevailing conditions such as desired pesticidal effect and duration, weather, target species, locus, mode of application, and the like. Similarly, an “insecticidally effective amount” may be used to refer to a “pesticidally effective amount” when the pest is an insect pest.

As used herein and unless otherwise indicated, the term “agriculturally acceptable carrier” “agriculturally acceptable excipient” or “agriculturally acceptable diluent” cover all adjuvants, inert components, dispersants, surfactants, tackifiers, binders, etc. that are ordinarily used in pesticide formulation technology; these are well known to those skilled in pesticide formulation.

In some embodiments, each of the individual compounds of the invention may also be administered by any convenient route, for example, orally, by infusion or bolus injection, or by absorption through epithelial or mucocutaneous linings (e.g., oral mucosa, rectal and intestinal mucosa, etc.), and may be administered together with another biologically active agent. Administration can be systemic or local. Various delivery systems are known, e.g., encapsulation in liposomes, microparticles, microcapsules, capsules, etc., and can be used to administer at least one of the compounds of the invention. Methods of administration of the individual compounds include but are not limited to intradermal, intramuscular, intraperitoneal, intravenous, subcutaneous, intranasal, epidural, oral, sublingual, intranasal, intracerebral, intravaginal, transdermal, rectal, pulmonary or topical, particularly to the ears, nose, eyes, or skin. The preferred mode of administration is left to the discretion of the practitioner, and will depend, in part, upon the site of the medical condition.

Each of the individual compounds to be administered can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule.

In some embodiments, when each of the individual compounds of the invention are administered intravenously, the compounds are in sterile isotonic aqueous buffered solutions. Where necessary, the individual compounds of the invention may also include a solubilizing agent. The individual compounds of the invention for intravenous administration may optionally include a local anesthetic such as lidocaine to ease pain at the site of the injection.

In one embodiment, individual compounds are supplied either together in a unit dosage form or separately. Regardless, compounds may be supplied, for example, as a dry lyophilized powder or water free concentrate in a hermetically sealed container such as an ampoule indicating the quantity of active agent. Where the compound or combination of compounds of the invention are to be administered by infusion, they can be dispensed, for example, with an infusion bottle containing sterile pharmaceutical grade water or saline. Where the compound or combination of compounds of the invention is administered by injection, an ampoule of sterile water for injection or saline can be provided so that the ingredients may be mixed prior to administration.

Compositions for oral delivery may be in the form of tablets, lozenges, aqueous or oily suspensions, granules, powders, emulsions, capsules, syrups, or elixirs, for example. Orally administered compositions may contain one or more optional agents, for example, sweetening agents such as fructose, aspartame or saccharin: flavoring agents such as peppermint, oil of wintergreen, or cherry: coloring agents; and preserving agents, to provide a pharmaceutically palatable preparation. Immediate release formulations for oral use include tablets or capsules containing the active ingredient(s) in a mixture with non-toxic pharmaceutically acceptable excipients. These excipients may be, for example, inert diluents or fillers (e g., sucrose, sorbitol, sugar, mannitol, microcrystalline cellulose, starches including potato starch, calcium carbonate, sodium chloride, lactose, calcium phosphate, calcium sulfate, or sodium phosphate): granulating and disintegrating agents (e.g., cellulose derivatives including microcrystalline cellulose, starches including potato starch, croscarmellose sodium, alginates, or alginic acid): binding agents (e.g., sucrose, glucose, mannitol, sorbitol, acacia, alginic acid, sodium alginate, gelatin, starch, pregelatmized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds of the invention.

For oral delivery, the compounds of the invention can be incorporated into a formulation that includes pharmaceutically acceptable carriers such as binders (e.g., gelatin, cellulose, gum tragacanth), excipients (e.g., starch, lactose), disintegrating agents (e.g., alginate, Primogel, and corn starch), and sweetening or flavoring agents (e.g., glucose, sucrose, saccharin, methyl salicylate, and peppermint). The formulation can be orally delivered in the form of enclosed gelatin capsules or compressed tablets. The capsules and tablets can also be coated with various coating known in the art to modify the flavors, tastes, colors, and shapes of the capsules and tablets. The carrier may be solid or a liquid, or both, and may be formulated with at least one compound described herein as the active compound which may contain from about 0.05% to about 95% by weight of the at least one active compound. Suitable oral formulations can also be in the form of suspension, syrup, chewing gum, wafer, elixir, and the like

The amount of each individual compounds of the invention to be administered will depend on the nature or severity of the symptoms, and can be determined by standard clinical techniques. In addition, in vitro or in vivo assays may optionally be employed to help identify optimal dosage ranges for each of the components of the combination. The precise dose of each component to be employed will also depend on the route of administration and the seriousness of the disease or disorder, and a practitioner can determine these doses based upon each patient's circumstances. In some embodiments, however, suitable dosage ranges for oral administration of each of the compounds of the invention are generally about 0.001 mg to 1000 mg of a compound of the invention per kilogram body weight. In specific embodiments of the invention, the oral dose for each compound of the present invention may be 0.01 mg to 100 mg per kilogram body weight, 0.1 mg to 50 mg per kilogram body weight, 0.5 mg to 20 mg per kilogram body weight, or 1 mg to 10 mg per kilogram body weight. In one embodiment, the oral dosage of each of the compounds of Formulae I to IV is at least about 1, 5, 10, 25, 50, 100, 200, 300, 400, or 500 mg/day up to as much as 600, 700, 800, 900, 1000 mg/day for three to fifteen days. Each of the compounds of Formulae I to IV may be given daily (e.g., once, twice, three times or four times daily) or less frequently (e g., once every other day, or once or twice weekly). The dosage amounts described herein refer to individual amounts administered. When more than one compound is administered, the preferred dosages correspond to the total amount of the compounds of the invention administered. The oral compositions described herein may contain from about 10% to about 95% active ingredient by weight.

In some embodiments, suitable dosage ranges for intravenous (i.v.) administration of each of the compounds of Formulae I to IV are 0.001 mg to 1000 mg per kilogram body weight, 0.01 mg to 100 mg per kilogram body weight, 0.1 mg to 50 mg per kilogram body weight, and 1 mg to 10 mg per kilogram body weight. In some embodiments, suitable dosage ranges for intranasal administration of each of the compounds of Formulae I to IV are from about 0.01 pg/kg body weight to 1 mg/kg body weight. Effective doses may be extrapolated from dose-response curves derived from in vitro or animal model test systems. Such animal models and systems are well known in the art.

### Identification of the Compound of Formula IV

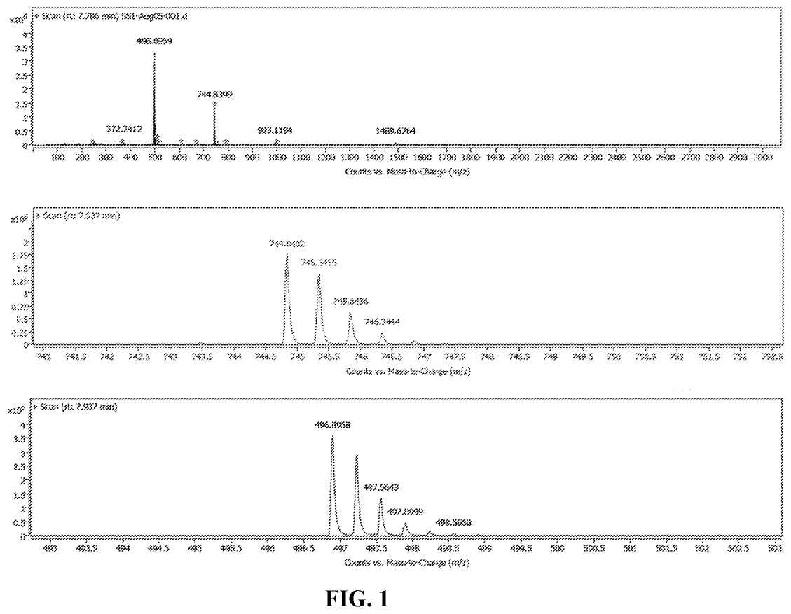

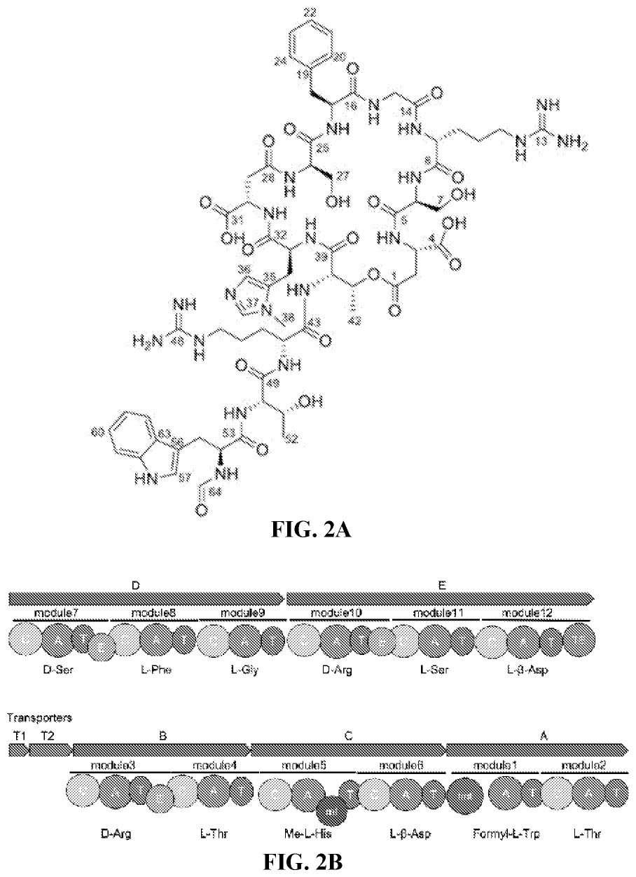

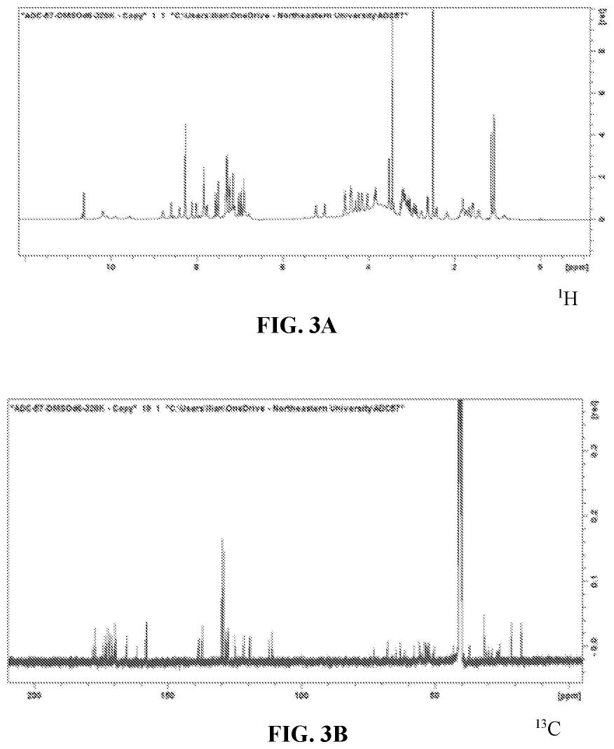

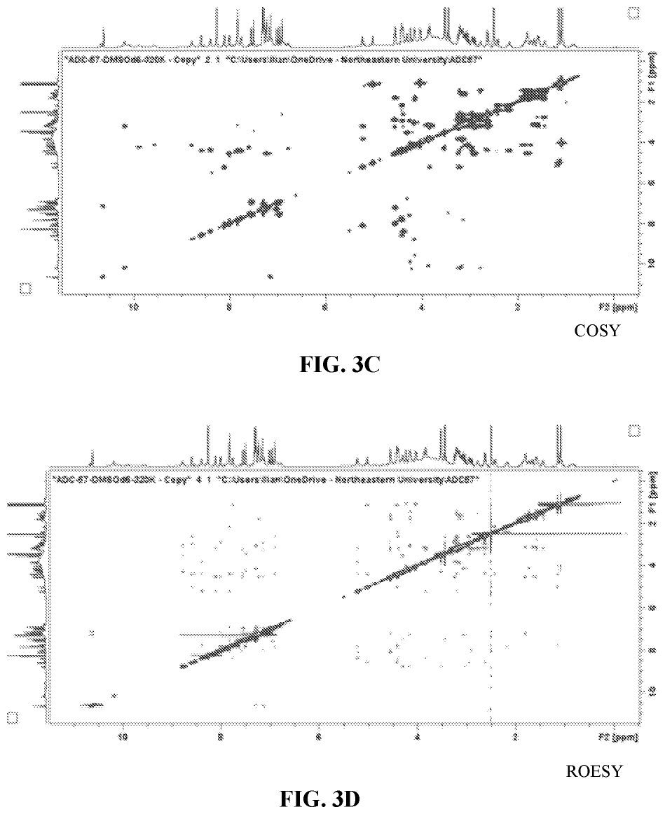

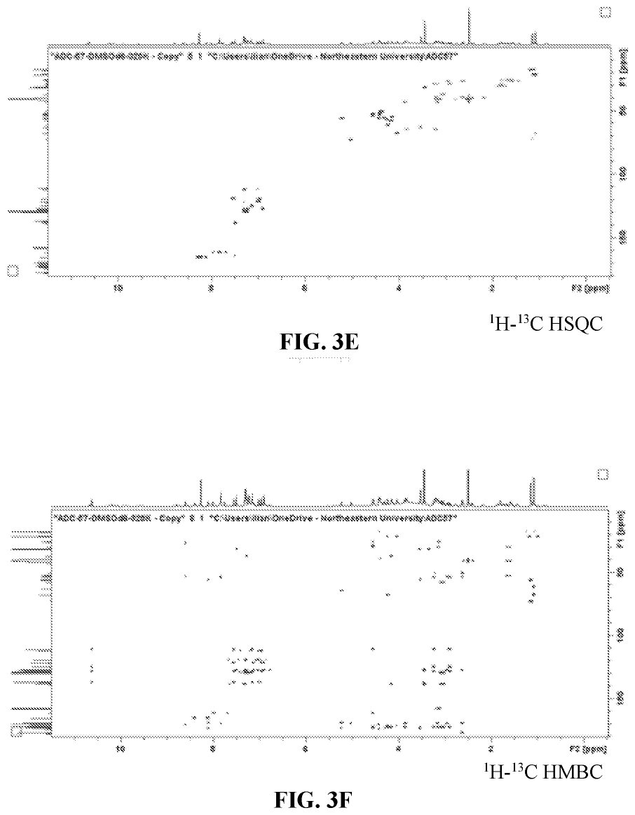

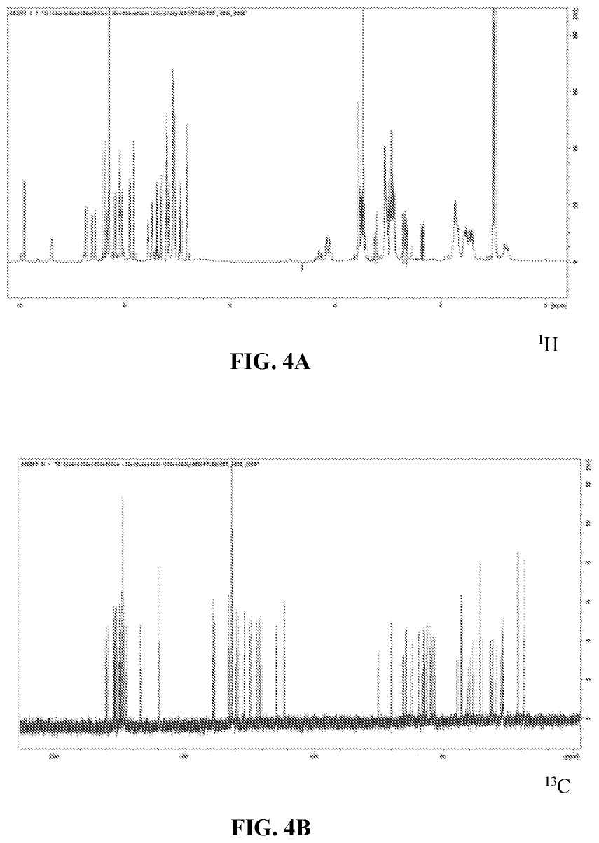

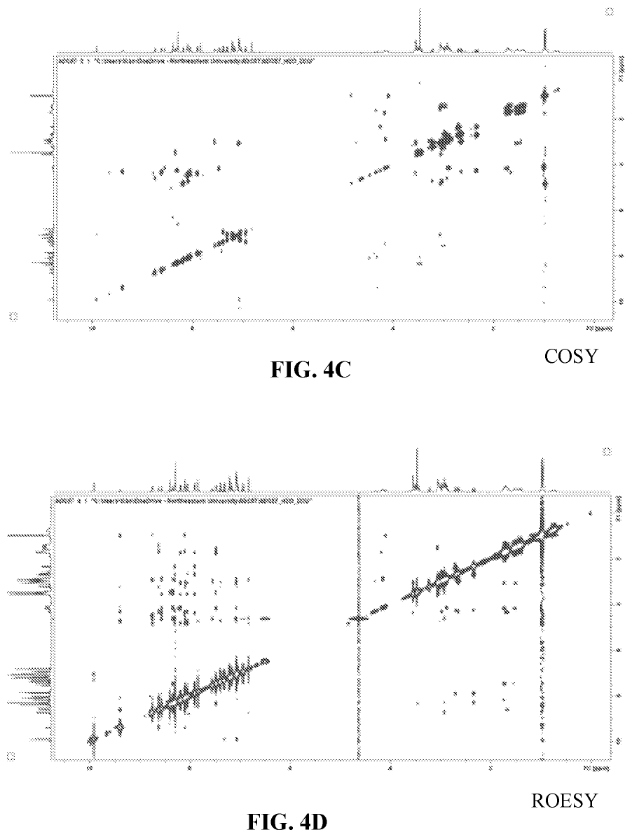

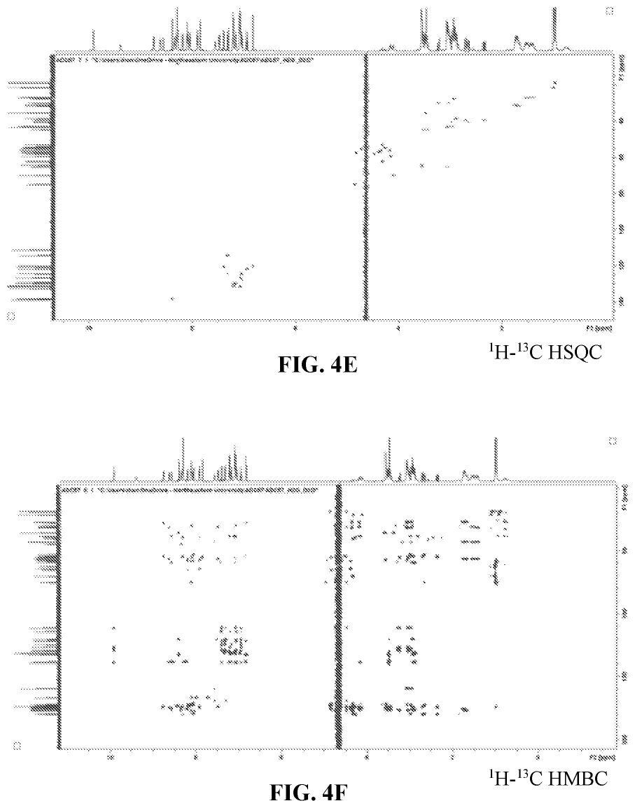

The inventors screened culture extracts from 58 strains of Photorhabdus and Xenorhabdus nematode symbionts against M. tuberculosis H37Rv mc²6020 expressing mCherry as a growth indicator. To avoid non-specifically acting compounds, the inventor used S. aureus HG003 as a counter screen to identify M. tuberculosis-specific compounds. A supernatant from P. noenieputensis DSM 25462 showed potent activity against M. tuberculosis but was inactive against S. aureus. The inventors optimized compound production by testing cultures grown in several different media. An extract from a culture grown in TNM-FH medium emulating insect haemolymph had a 2- to 4-fold higher activity as compared to Luria-Bertani Broth (LBB) and Tryptic Soy Broth (TSB). To isolate the active compound, the supernatant was concentrated 200-fold and fractionated using high-performance liquid chromatography (HPLC) and subjected to bio-assay guided purification to identify the active fraction. 101 fractions were collected, and activity was concentrated in fractions 57 to 68. Active fractions were further subjected to HPLC for final purification. High-resolution electrospray ionization-mass spectrometry (HR-ESI-MS) analysis revealed the molecular mass of the active compound ([M+H]+=1488.68), which did not match any known compounds in Antibase. FIG. 1 illustrates a MS spectrum of the compound of Formula IV. The structure of the compound was determined by nuclear magnetic resonance (NMR) and MS spectroscopic analyses. See FIGS. 2A and 2B, FIGS. 3A to 3F, FIG. 4A to 4F. Tables 1 and 2 below summarize the data for the compound of Formula IV.

TABLE 1
¹H, ¹³C, and ¹⁵N NMR (700/175/70 MHz)
chemical shift in DMSO-d₆ at 320K.

| Amino acid | | | |
|---|---|---|---|
| residue | Position | δ_{c}/δ_{N} | δ_{H} (mult., J in Hz) |
| β-Asp | 1 | 176.1 | — |
| | 2 | 38.3 | 2.62/3.20 (2H, ovl^{a}) |
| | 3 | 49.5 | 4.38 (1H, ovl^{a}) |
| | 4 | 168.7 | — |
| | 3-NH | 122.6 | 8.39 (1H, d, 7.9) |
| Ser | 5 | 168.3 | — |
| | 6 | 54.9 | 5.22 (1H, dt, 5.6, 8.3) |
| | 7 | 65.6 | 3.20/3.82 (2H, ovl^{a}) |
| | 6-NH | 116.1 | 8.11 (1H, d, 8.6) |
| Arg | 8 | 172.5 | — |
| | 9 | 51.7 | 4.54 (1H, ovl^{a}) |
| | 10 | 29.0 | 1.80 (2H, m) |
| | 11 | 24.8 | 1.58 (1H, ovla)/ |
| | | | 1.66 (1H, m, 6.9, 7.2) |
| | 12 | 40.1 | 3.14 (2H, ovl^{a}) |
| | 13 | 156.9 | — |
| | 9-NH | 116.0 | 7.22 (1H, ovl^{a}) |
| Gly | 14 | 168.8 | — |
| | 15 | 42.2 | 3.15/3.85 (2H, ovl^{a}) |
| | 15-NH | 105.2 | 10.09 (1H, broad) |
| Phe | 16 | 171.6 | — |
| | 17 | 56.9 | 4.15 (1H, ovla) |
| | 18 | 36.1 | 3.04 (1H, d, 9.3)/ |
| | | | 3.07 (1H, ovl^{a}) |
| | 19 | 137.5 | — |
| | 20/24 | 128.7 | 7.28 (1H, ovl^{a}) |
| | 21/23 | 128.2 | 7.30 (1H, ovl^{a}) |
| | 22 | 126.4 | 7.23 (1H, t, 7.0) |
| | 17-NH | 119.5 | 8.78 (1H, broad) |
| Ser | 25 | 172.4 | — |
| | 26 | 54.7 | 4.30 (1H, dt, 5.2, 4.7) |
| | 27 | 61.9 | 3.52 (2H, d, 4.3) |
| | 26-NH | 113.1 | 6.77 (1H, broad) |
| β-Asp | 28 | 168.6 | — |
| | 29 | 38.9 | 2.17 (1H, ovl^{a})/ |
| | | | 2.42 (1H, d, 14.2) |
| | 30 | 51.1 | 4.40 (1H, ovl^{a}) |
| | 31 | 176.9 | — |
| | 30-NH | 127.1 | 7.83 (1H, ovl^{a}) |
| His | 32 | 169.0 | — |
| | 33 | 52.4 | 4.41 (1H, ovl^{a}) |
| | 34 | 25.9 | 2.63/2.94 (2H, ovl^{a}) |
| | 35 | 126.6 | — |
| | 36 | 126.4 | 6.89 (1H, s) |
| | 37 | 137.4 | 7.49 (1H, s) |
| | 33-NH | 121.8 | 8.60 (1H, d, 7.1) |
| | 38 | 30.6 | 3.44 (3H, s) |
| Thr | 39 | 169.9 | — |
| | 40 | 55.0 | 4.42 (1H, ovl^{a}) |
| | 41 | 71.9 | 5.02 (1H, m) |
| | 42 | 16.8 | 1.14 (3H, d, 6.3) |
| | 40-NH | 106.0 | 7.75 (1H, d, 9.3) |
| Trp | 53 | 171.6 | — |
| | 54 | 52.8 | 4.55 (1H, ovla) |
| | 55 | 27.7 | 2.89/3.23 (2H, d, 3.9) |
| | 56 | 110 | — |
| | 57 | 123.9 | 7.15 (1H, d, 2.2) |
| | 58 | 136.0 | — |
| | 59 | 111.1 | 7.29 (1H, ovla) |
| | 60 | 120.7 | 7.01 (1H, t, 7.7) |
| | 61 | 118.4 | 6.96 (1H, dd, 7.4, 7.5) |
| | 62 | 118.2 | 7.55 (1H, d, 7.9) |
| | 63 | 127.3 | — |
| | 64 | 160.6 | 7.82 (1H, s) |
| | 54-NH | 125.9 | 8.00 (1H, d, 8.4) |
| | 57-NH | 129.9 | 10.62 (1H, apr^{b} s) |

^{a}Coupling patterns and constants are not identified due to signal overlapping of different protons.
^{b}Apparently singlet resulting from current instrumentation resolution.

TABLE 2
Retention times (t_{R}, min) of the FDLA and GITC
derivatives for the compound of Formula IV.

| Amino acid | M.W. | t_{RL} | t_{RD} | order | Assignment |
|---|---|---|---|---|---|
| Asp | 427 | 10.91 | 11.07 | L → D | L |
| Ser | 399 | 10.83 | 10.83 | — | L/D |
| | | 11.02 | 11.02 | | |
| Arg | 468 | 9.34 | 9.66 | L → D | D |
| Phe | 459 | 13.09 | 14.25 | L → D | L |
| methylhistidine | 463 | 9.09 | 9.43 | L → D | L |
| Thr | 413 | 10.79 | 11.78 | L → D | L |
| Trp | 498 | 12.92 | 13.63 | L → D | L |
| Thr (the compound | 508 | 9.49 | — | — | L-Thr |
| of Formula IV) | | | | | |
| L-Thr | 508 | 9.45 | — | — | — |
| D-allo-Thr | 508 | 9.30 | — | — | — |

The peptidic nature of the compound of Formula IV was evident from the ¹H and ¹³C NMR spectra showing the presence of the α-protons and amide carbonyl signals. A detailed 2D NMR spectroscopic analysis (COSY, HSQC, HMBC, and ROESY) determined the composition and sequence of the amino acid residues in the peptide. The discrimination between α and β-aspartic acids was particularly challenging. The 1,1-ADEQUATE in combination with ROESY and HMBC experiments revealed the presence of two β-aspartic acids in the molecule. The absolute configuration of the amino acids was determined by Marfey's analysis. This structurally novel antibiotic is a cyclic depsipeptide composed of 12 L- and D-amino acids with an N-formylated branch.

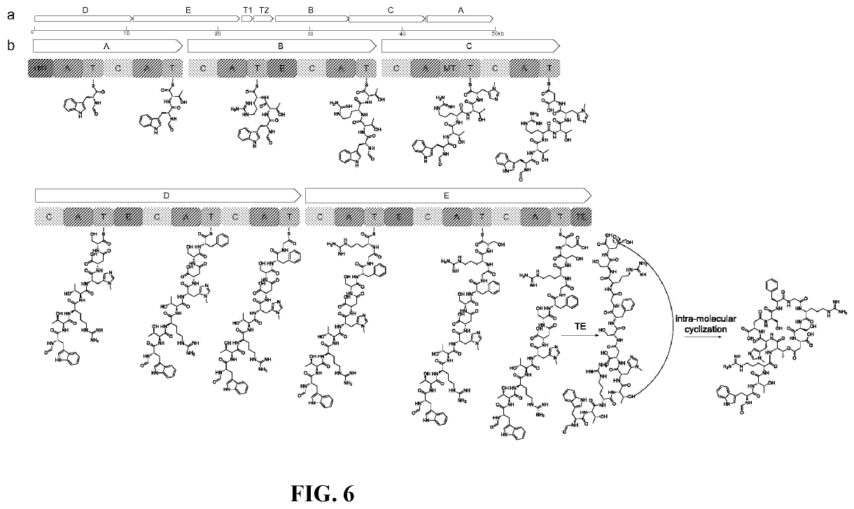

The biosynthetic gene cluster (BGC) of the compound of Formula IV was determined using bioinformatic analysis of the genome. The genome was sequenced by combination of Nanopore and Illumina reads [Microbial Genome Sequencing Center (MiGS; Pittsburgh, PA)] and assembled into two contigs with a total size of 5.5 Mb. AntiSMASH 5.0 was used to analyze BGCs in the contigs. As the main building block of the compound of Formula IV is the amino acid, the non-ribosomal peptide synthetase (NRPS) with adjacent tailoring enzymes such as formyltransferase and methyltransferase was deduced to be responsible for the biosynthesis of the compound of Formula IV. The BGC of the compound of Formula IV was identified as a non-ribosomal peptide synthase with a core BGC spanning 49.6 Kb. See FIG. 2B and FIG. 6. The number of NRPS modules was in accordance with the number of amino acids in the compound of Formula IV. The BGC has five core type I NRPS genes containing 12 linear modules. Adenylation domains were used to predict amino acid substrate specificity using AntiSMASH and Prism default settings. A formyltransferase was identified from module 1, which is consistent with formylation of the tryptophan amide. Similarly, a methyltransferase was identified from module 5, consistent with the N-methylation of the histidine moiety. Both arginine loading modules (modules 3 and 10) contain an epimerization domain. Modules 7 and 11 were predicted to incorporate serine moieties while an epimerization domain was only found in module 7. These results are consistent with Marfey's analysis and suggest the serine in module 7 has a D-configuration (Table 2). The GC content of the compound of Formula IV BGC is 46%, and there is no identical BGC in other bacterial species based on AntiSMASH search.

### Structural Analysis of the Compound of Formula IV

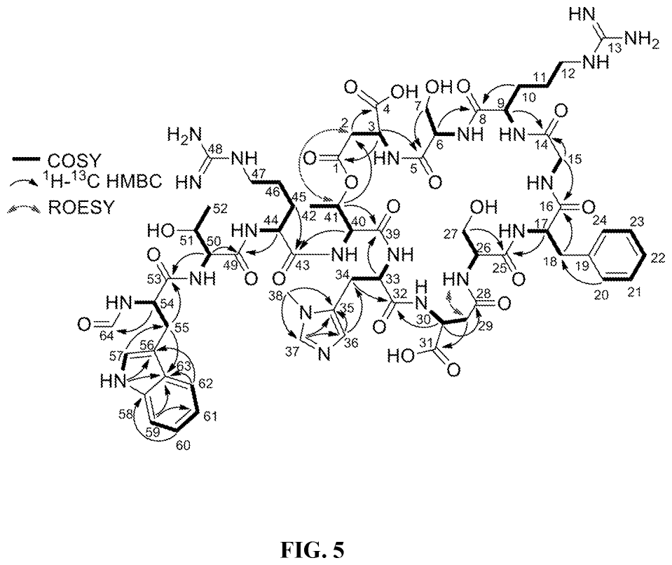

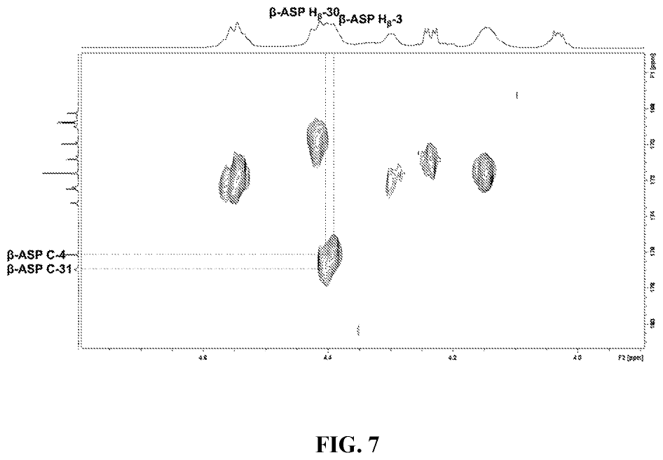

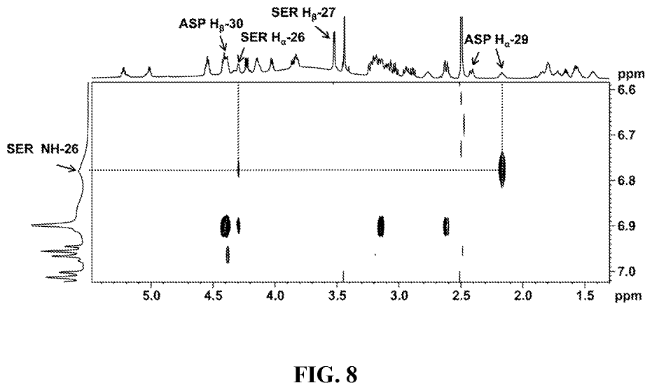

High-resolution ESI-MS analysis of the compound of Formula IV showed the following data: m/z 1488.68 [M+H]⁺, calcd for C₆₄H₉₀N₂₁O₂₁⁺, 1488.66 (FIG. 1). The ¹H and HSQC NMR data recorded in DMSO-d₆ showed numerous amide proton signals (δ_{H} 7.22-10.09) and α-amino methines (δ_{H} 4.13-5.22), indicative of a peptidic structure (Table 1). This was supported by the presence of carbonyl carbons in ¹³C NMR spectrum. The compound of Formula IV showed unusual signals that are not observed in standard amino acids. The singlet methyl signal at δ_{H} 3.44 indicated the presence of N-methylated residue. N-formyl modification was readily deduced by distinctive signals at δ_{C/H} 160.6/7.82. Further comprehensive analysis of 2D NMR data (COSY, HMBC, and ROESY) enabled the identification of 11 standard amino acids, including two aspartic acids, two serines, two arginines, two threonines, one glycine, one phenylalanine, one histidine, and one tryptophan (FIG. 5). The strong HMBC correlations from the singlet methyl group H-38 (δ_{H} 3.44) to C-35 (dc 126.6) and C-37 (δ_{C} 137.4) positioned the N-methyl group C-38 (δ_{C} 30.6) at the π position of the histidine. Likewise, the formyl group C-64 (δ_{C} 160.6) was shown to be positioned at the amino group of tryptophan (54—NH) by the HMBC correlation from the methine proton H-54 (δ_{H} 4.55) to C-64. The connectivity of the identified residues was determined on the basis of HMBC correlations from α-protons to amide carbonyls of adjacent residues. It still remained ambiguous whether aspartic acid was connected via C-1 (backbone) or C-4 (side chain) because the HMBC experiment cannot differentiate two-bond and three-bond correlations. The 1,1-ADEQUATE spectra was measured to address this issue. Correlations from H-30 (δ_{H} 4.40) to C-31 (δ_{C} 176.9) and from H-3 (δ_{H} 4.38) to C-4 (δ_{C} 168.7) were observed (FIG. 7), indicating the connection via the side chain of aspartic acid. This result strongly showed that two aspartic acids in the compound are β-aspartic acids and was further supported by ROESY and HMBC experiments. A ROESY correlation between H-29 (δ_{H} 2.17) and NH-26 (δ_{H} 6.77) suggested a β-aspartic acid linkage between a serine moiety and a methylated histidine moiety (FIG. 8). Another β-aspartic acid linkage between the terminal aspartic acid and threonine through an ester bond was established by the long-range HMBC correlation from H-41 (δ_{H} 5.02) to C-2 (δ_{C} 38.3). This connection was further supported by the ROESY correlation between H-2 (δ_{H} 4.38) and H-41 observed in the spectrum acquired in 4% D₂O in H₂O. The planar structure of the compound of Formula IV was therefore characterized as a cyclic depsipeptide with a N-formylated branch.

The stereochemistry of chiral centers present at α and β-carbons were assigned by applying derivatization methods coupled with chromatographic analysis. The advanced Marfey's method using L- and D-FDLA (1-fluoro-2,4-dinitrophenyl-5-leucinamide) established the absolute configurations of amino acids: L-Asp, L-Ser, D-Ser, D-Arg, L-Phe, L-methylhistidine, L-Thr, and L-Trp (Table 2). The remaining chiral centers at β-carbons were determined by the LC/MS analysis of the GITC (2,3,4,6-tetra-O-acetyl-β-D-glucopyranosyl isothiocyanate) derivatives. The regiochemistry of L- and D-Ser in the compound of Formula IV relied on A domain analysis of the biosynthetic gene cluster.

### Spectrum of Activity

Notably, the compound of Formula IV is highly potent against M. tuberculosis, with a minimum inhibitory concentration (MIC) of 0.25 μg ml⁻¹ (Table 3). Standard treatment of M. tuberculosis infection requires four different antibiotics administered over a 6-9 month period. This prolonged treatment poses a serious risk to the human microbiome, potentiating dysbiosis and evolution of resistance in off target bacteria. With this in mind, the inventors tested the compound of Formula IV against commensal bacteria including Lactobacillus sp. and Bacteroides sp., finding no activity (Table 3). Additionally, the compound of Formula IV showed no toxicity against HepG2, FaDu, and HEK293 human cell lines (Table 3). Taken together, these results demonstrate that the compound of Formula IV is highly selective against M. tuberculosis. This selectivity suggested action against a target specifically present in Mycobacteria and absent from human cells.

TABLE 3
Spectrum of the compound of Formula IV

| | |
|---|---|
| | MIC |
| Strain | (μg/ml) |

Pathogenic bacteria (MIC)

| | |
|---|---|
| Mycobacterium tuberculosis H37Rv mc²6020 mCherry | 0.0625 |
| Mycobacterium tuberculosis H37Rv mc²6020 | 0.25 |
| Mycobacterium tuberculosis H37Rv mc²6020 ΔbacA | 16 |
| Mycobacterium tuberculosis H37Rv mc²6020 gyrA G88S | 64 |
| Mycobacterium tuberculosis H37Rv mc²6020 gyrA G88C | 128 |
| Mycobacterium smegmatis mc²155 | 8 |
| Mycobacterium abscessus ATCC 19977 | 16 |
| Staphylococcus aureus HG003 | 128 |
| Escherichia coli WO153 | 0.0625 |
| Escherichia coli ΔtolC^{a} | 0.25 |
| Escherichia coli ATCC25922 | 8 |
| Escherichia coli BW25113 | 16 |
| Escherichia coli MG1655 | 16 |
| Escherichia coli ΔSbmA^{a} | 128 |
| Klebsiella pneumoniae ATCC 700603 | 32 |
| Clostridium perfringens KLE 2523^{b, c} | 64 |
| Enterococcus faecalis KLE 2341^{b, c} | >128 |
| Acinetobacter baumannii ATCC 17978 | 128 |
| Salmonella enterica KLE 2601^{b, c} | 128 |
| Pseudomonas aeruginosa PA01 | >128 |

Symbiotic bacteria (MIC)

| | |
|---|---|
| Lactobacillus reuteri LTH5448^{b} | >128 |
| Lactobacillus paracasei KLE 2504^{b, c} | >128 |
| Streptococcus parasanguinis KLE 2509^{b, c} | >128 |
| Bacteroides fragilis KLE 2244^{b, c} | 64 |
| Bacteroides stercoris KLE 2537^{b, c} | >128 |
| Veillonella ratti KLE 2365^{b, c} | >128 |

Human cell line (IC50)

| | |
|---|---|
| HepG2 | >128 |
| FaDu | >128 |
| HEK293 | >128 |

^{a}Keio collection mutants.
^{b}Cultivated under anaerobic conditions.
^{c}Human stool isolate, K.L. laboratory collection.

### Animal Efficacy

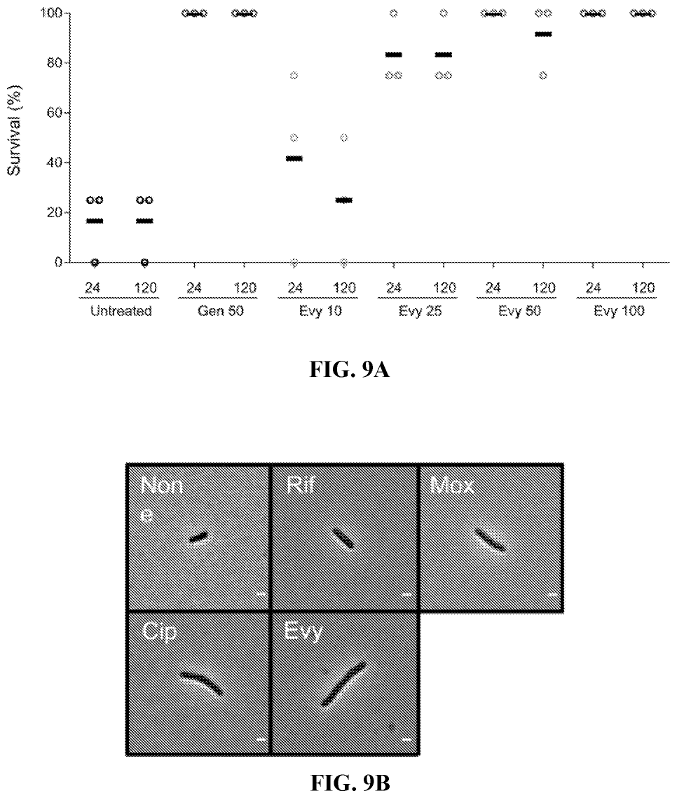

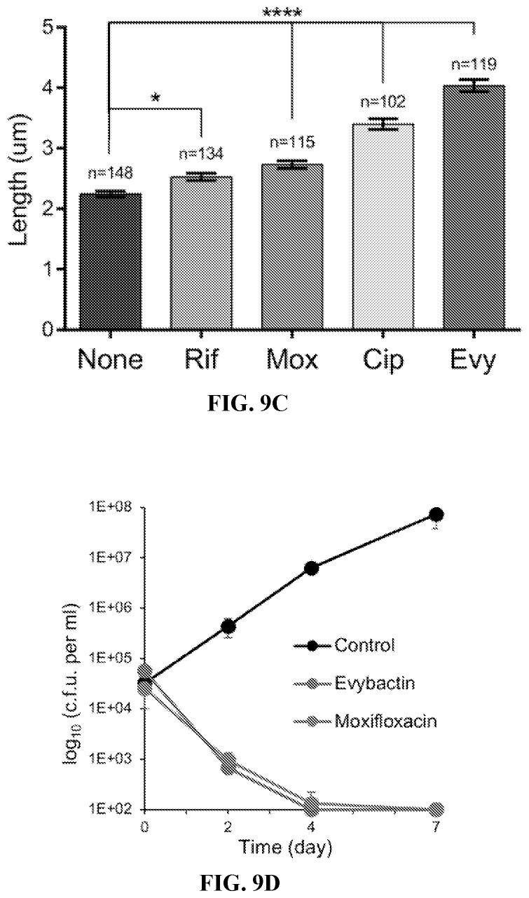

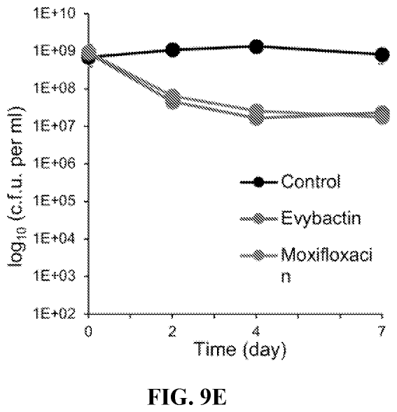

The inventors next tested the compound of Formula IV in a simple model of septicemia infection with E. coli to evaluate its potential for activity in vivo. First, to assess the toxicity of the compound of Formula IV in a mouse, the compound of Formula IV was administrated intraperitoneally at 100 mg kg⁻¹ and survival was observed for 24 hours. There were no indications of toxicity. Since the compound of Formula IV showed a relatively low MIC against E. coli ATCC25922, the inventors used this strain for a preliminary animal study. Mice were infected with E. coli ATCC25922 intraperitoneally for 1 hour followed by intraperitoneal administration of the compound of Formula IV. A single dose of 25 mg kg⁻¹ the compound of Formula IV showed significant efficacy and a 100 mg kg⁻¹ dose of the compound of Formula IV completely protected mice from E. coli infection, while 83% of untreated control animals died within 24 h (FIG. 9A).

### Mechanism of Selectivity

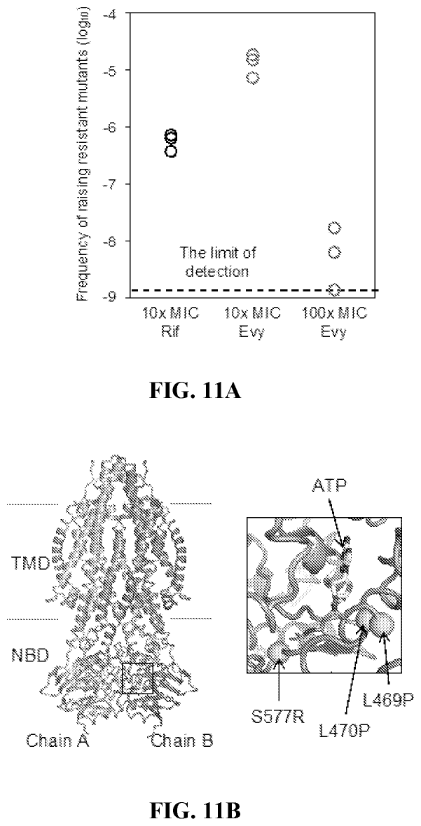

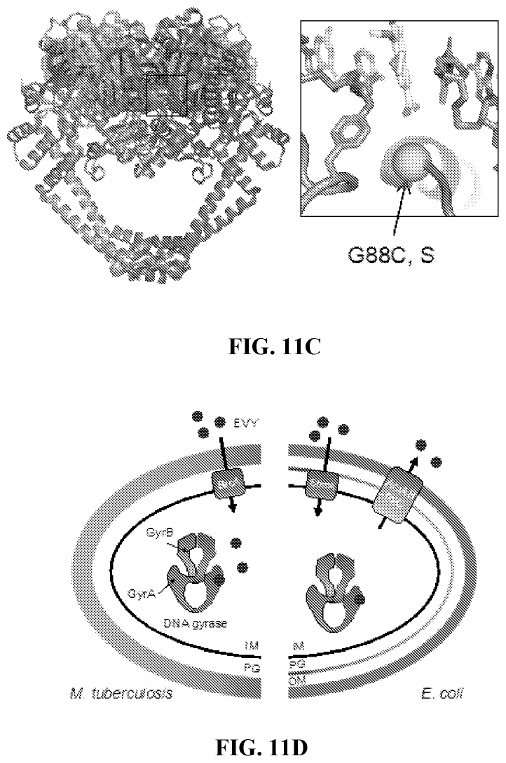

To identify the target of the compound of Formula IV, the inventors isolated resistant mutants from M. tuberculosis H37Rv mc²6020. M. tuberculosis cells were seeded onto 7H9 nutrient agar medium containing 4×MIC of the compound of Formula IV and gave rise to the compound of Formula IV-resistant mutants with a frequency of 7.2×10⁻⁶ to 1.6×10⁻⁵ (FIG. 11A). The inventors sequenced the whole genome of three spontaneous mutants and found that all of the strains carry mutations (L469P, L470P and S577R) in the membrane transporter bacA (Rv1819c) (FIG. 11B). All the spontaneous evybactin-resistant mutants, as well as a bacA deletion mutant, had an increased MIC (8-16 μg ml⁻¹, or 32-64×MIC) (Table 3).

BacA is annotated as a vitamin B12 transporter, however, a recent study proposed that BacA serves as a multi-solute ABC-type transporter for hydrophilic molecules. The compound of Formula IV is a highly hydrophilic compound whose solubility in water is more than 40 mg ml⁻¹. This property suggests that the compound of Formula IV uses BacA to penetrate into M. tuberculosis cells. Notably, mutations in the resistant mutants mapped to the nucleotide binding domain (NBD) of BacA. A previous study demonstrated that a specific mutation in the BacA (E576G) led to the loss of ATPase activity and eliminated the transport function of BacA. This result suggests that BacA is non-functional in the M. tuberculosis mutants resistant to the compound of Formula IV.

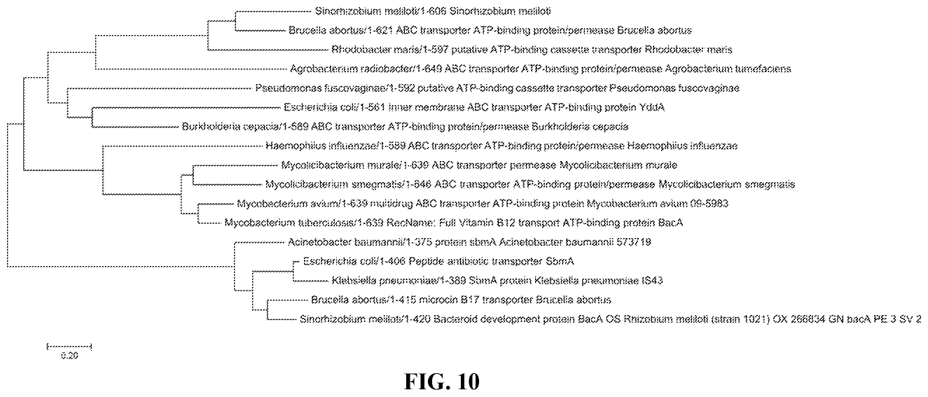

BacA homologues are sparsely distributed among other bacteria and are found in E. coli (SbmA, which serves as a peptide antibiotic microcin transporter) (FIG. 10). The inventors took advantage of this homology to test the possible role of SbmA in the susceptibility of E. coli to the compound of Formula IV. The compound of Formula IV MIC for wild type E. coli is 16 μg ml⁻¹, considerably higher as compared to M. tuberculosis. Susceptibility to the compound of Formula IV further decreases in an E. coli mutant with a knockout in sbmA (Table 3). Notably, the inventors found that E. coli WO153 with a compromised penetration barrier is highly sensitive to the compound of Formula IV, with an MIC of 0.0625 μg ml⁻¹ (Table 3). E. coli WO153 expresses less lipopolysaccharide (LPS) and lacks the outer membrane porin TolC that serves as a docking port for multidrug resistance pumps. This observation suggests that outer membrane permeability and/or efflux restrict penetration of the compound of Formula IV into E. coli. The MIC for an E. coli tolC deletion mutant was 0.25 μg ml⁻¹, 4 times higher as compared to the E. coli WO153 strain, and 64 times lower than in the wild type. This finding suggests that both the outer membrane barrier and efflux across it contribute to the high resistance of wild type E. coli to the compound of Formula IV. Taken together, these results suggest that in E. coli, the compound of Formula IV is transported into the cell by SbmA, but efficiently effluxed by TolC-dependent MDRs (FIG. 11D). The Gram-positive M. tuberculosis lacks a comparable restrictive penetration barrier, and as a result is sensitive to the compound of Formula IV, which is smuggled into the cell by BacA. This consideration explains the selectivity of the compound of Formula IV against M. tuberculosis and also explains resistance of gut commensal bacteria that lack a BacA-type transporter to the compound of Formula IV.

### The Target of the Compound of Formula IV

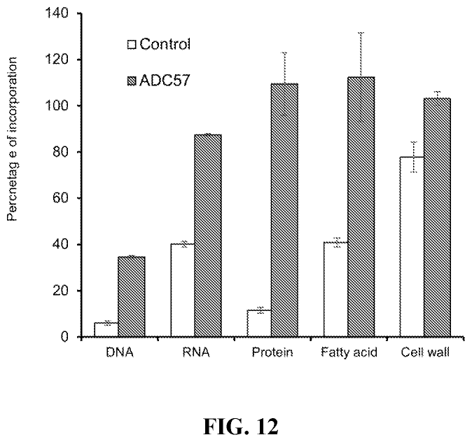

Considering that BacA is non-essential and likely only serves as the transporter for the compound of Formula IV, the inventors assumed that the true target is located in the cytoplasm. A label incorporation assay revealed that the compound of Formula IV inhibits DNA synthesis but has less of an effect on RNA, protein and fatty acid synthesis (FIG. 12). In agreement with this result, M. tuberculosis cells treated with the compound of Formula IV were approximately 2 times longer compared to nontreated cells. This morphological change is typical for inhibition of DNA synthesis (FIG. 9B and FIG. 9C).

To identify the true target of the compound of Formula IV, the inventors selected M. tuberculosis mutants in the presence of a high concentration of the compound, 25 μg ml⁻¹ (100×MIC) to avoid selection for bacA mutants (MIC 8 μg ml⁻¹, 32×MIC). This treatment resulted in the selection of mutants highly resistant to the compound of Formula IV, at a frequency of 6.1×10⁻⁹ to 1.7×10⁻⁸ to (FIG. 11A). The inventors sequenced the whole genome of two high level the compound of Formula IV-resistant mutants and found that they harbor G88S or G88C mutations in gyrA (Rv0006), which codes for DNA gyrase subunit A (FIG. 11C). The inventors confirmed that the high level of the compound of Formula IV resistance is due to mutations in gyrA by constructing gyrA recombinant M. tuberculosis mutants in a clean background (Table 3).

To test whether there is cross-resistance between the compound of Formula IV and moxifloxacin, a known DNA gyrase inhibitor, the inventors isolated spontaneous moxifloxacin-resistant mutants of M. tuberculosis (GyrA D94N). The inventors then evaluated the susceptibility of the compound of Formula IV and moxifloxacin against the compound of Formula IV-resistant mutants (GyrA G88C and G88S) and the moxifloxacin-resistant mutant (GyrA D94N). The GyrA G88C mutation is known to confer fluoroquinolone resistance to M. tuberculosis and, in agreement with this finding, GyrA G88C mutant is resistant to moxifloxacin (Table 4). However, contrary to our expectations, the GyrA G88S mutation makes M. tuberculosis more susceptible to moxifloxacin, whereas the GyrA D94N mutation did not have any effect on the compound of Formula IV susceptibility (Table 4). These results suggest that the two structurally distinct compounds bind differently to DNA gyrase.

TABLE 4
MIC of DNA gyrase inhibitors against
M. tuberculosis GyrA mutants

| | MIC (μg ml⁻¹) | |
|---|---|---|
| | The compound | |
| Strain | of Formula IV | Moxifloxacin |
| M. tuberculosis H37Rv mc²6020 | 0.25 | 0.125 |
| GyrA G88C mutant^{a} | >128 | 4 |
| GyrA G88S mutant^{a} | 64 | 0.0156 |
| GyrA D94N mutant^{b} | 0.25 | 2 |

^{a}GyrA recombinant mutant.
^{b}Spontaneous moxifloxacin-resistant mutants.

DNA gyrase poisons are bactericidal antibiotics. The inventors therefore evaluated the killing ability of the compound of Formula IV. The compound was highly bactericidal against exponentially growing and stationary M. tuberculosis with activity similar to moxifloxacin, which is often used as a second-line antibiotic for extended multidrug-resistant mutants of M. tuberculosis (FIG. 9D, FIG. 9E, and Table 3).

To verify gyrase as the target of the compound of Formula IV in M. tuberculosis, the inventors conducted in vitro biochemical assays using the purified M. tuberculosis enzyme (MtbGyrase). Type II topoisomerases, including DNA gyrase, regulate DNA supercoiling and chromosome entanglements by creating a transient double stranded break in one DNA duplex and passing a second double-stranded segment through the break. Bacterial type II topoisomerase poisons, such as moxifloxacin, corrupt this strand passage process, leading to persistent double-stranded DNA breaks and cell death.

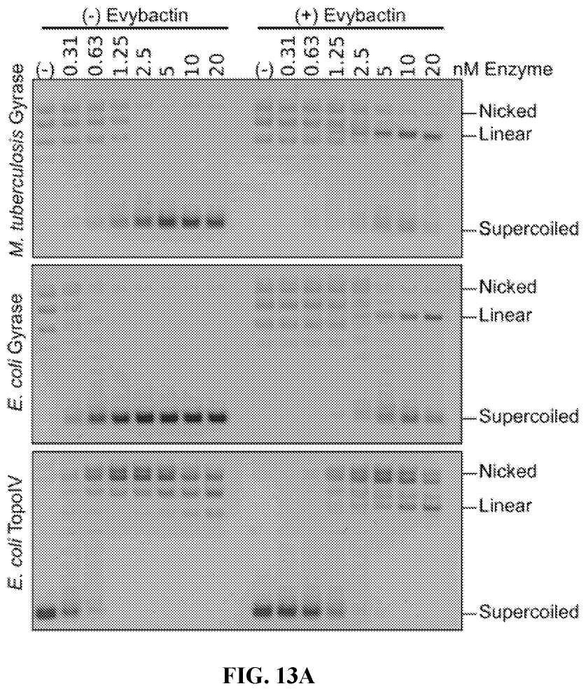

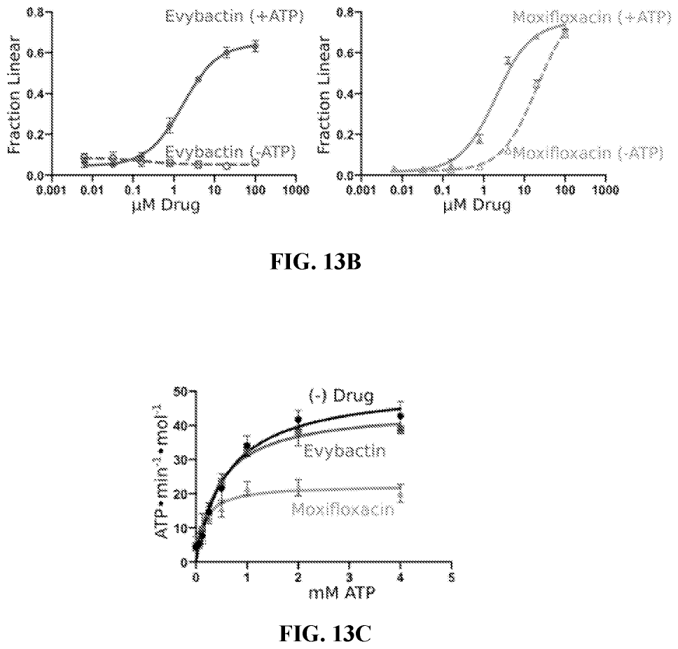

To test the ability of the compound of Formula IV to induce DNA cleavage by MtbGyrase, the inventors conducted cleavage assays by titrating the enzyme against a fixed amount of the compound and plasmid, quenching with EDTA and SDS/Proteinase-K, and separating the reactants by native agarose gel electrophoresis. The compound of Formula IV not only inhibited the supercoiling activity of MtbGyrase but also induced DNA cleavage, as evidenced by the appearance of both supercoiled and linear cleavage products at higher enzyme concentrations (FIG. 13A, top panel). Since E. coli WO153 and tolC deletion strains were susceptible to the compound of Formula IV, the inventors further assayed the ability of the agent to induce cleavage by the two known E. coli type II topoisomerases, gyrase and topoisomerase IV (topo IV). The compound of Formula IV stimulated DNA cleavage by both enzymes to an extent similar to that of MtbGyrase (strong cleavage visible at 5 nM enzyme for both species) (FIG. 13A, middle and bottom panels); however, the compound was a more potent inhibitor of DNA supercoiling by gyrase than of DNA supercoil relaxation by topo IV. These data demonstrate that, similar to the fluoroquinoline antibiotics, the compound of Formula IV is a general poison of bacterial type IIA topoisomerases, with a preference for gyrase as compared to topo IV.

Since the compound of Formula IV and moxifloxacin exhibited similar killing of M. tuberculosis, and both act as gyrase poisons, the inventors sought to compare the effect of the two compounds on MtbGyrase in vitro. Using a fixed amount of MtbGyrase and titrating moxifloxacin in the presence of ATP, the inventors observed robust levels of DNA cleavage (IC₅₀≈1 μM) (FIG. 13B). The compound of Formula IV stimulated cleavage with comparable efficiency in the presence of ATP, with an IC₅₀ also close to 1 μM. Interestingly, little to no cleavage was observed for the compound of Formula IV in the absence of ATP, whereas moxifloxacin produced robust nucleotide-independent cleavage at a concentration of 5-10 μM. To further investigate the differences between the mechanisms of action for moxifloxacin and the compound of Formula IV, the inventors tested the effect of both compounds on the DNA-stimulated ATPase activity of MtbGyrase. Although the compound of Formula IV has only a slight effect on the overall rate (vₘₐₓ) of MtbGyrase's ATPase activity (51.6±10.2 ATP·s⁻¹ per enzyme vs 45±7.2 ATP·s⁻¹ per enzyme), the addition of moxifloxacin resulted in a roughly two-fold reduction of ATP consumption (22.2±6 ATP·s⁻¹ per enzyme) (FIG. 13C). As suggested by the disparate effects of the D94N and G88S mutants on MIC values for the compound of Formula IV and moxifloxacin (Table 4), the strict dependence on ATP by the compound of Formula IV, as well as its negligible effect on ATPase activity, suggested that the mechanism of action for the molecule is distinct from that of fluoroquinolones.

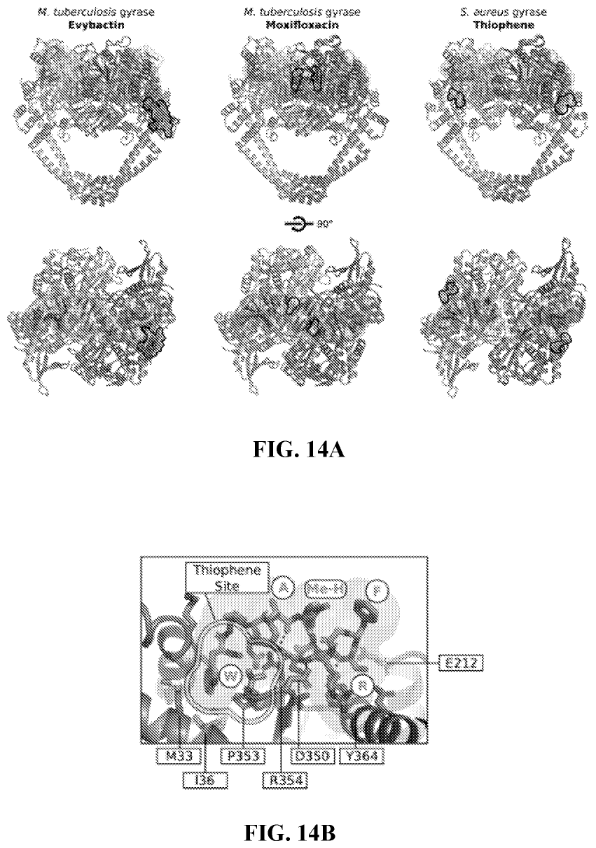

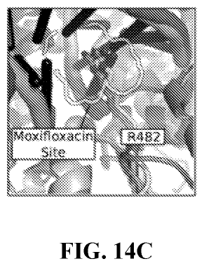

To more precisely determine the compound of Formula IV mechanism of action, the inventors co-crystallized a portion of MtbGyrase bound to the compound and a singly-nicked duplex DNA substrate (Methods). For this, the inventors used a fusion construct comprising the DNA binding and cleavage region of M. tuberculosis gyrase (MtbGyrBA_{core}) that the inventors had generated previously to study cleavage complexes of the enzyme bound to fluoroquinolone poisons, but harboring a Y129F mutation, which prevents the enzyme from cleaving DNA. The resultant structure revealed that the compound of Formula IV binds at a site distal to the fluoroquinolone binding pocket (FIG. 14A, Table 5). The macrocycle depsipeptide portion of the compound of Formula IV engages a winged-helix domain in GyrA using an “edge-on” conformation in which the protein is engaged by only one face of the compound (FIG. 14B). Edge-on binding poses are among the most common types of protein macrocycle interaction and, along with “face-on” and “compact” binding modes where the macrocycle lays flat or within a binding pocket, account for most observed geometries of macrocycles bound to proteins.

TABLE 5
Data collection and structure refinement statistics

| | MtbGyrase the Compound of Formul IV | |
|---|---|---|
| Waivelength | | 0.9201 |
| Resolution range | 45.18-2.9 (3.004-2.9) | |
| Space group | P 21 21 21 | |
| Unit cell | 83.075 105.088 | |
| | 250.183 90 90 90 | |
| Total reflections | 147996 (15040) | |
| Unique reflections | 36794 (4867) | |
| Multiplicity | 4.0 (4.0) | |
| Completeness (%) | 99.65 (99.69) | |
| Mean I/sigma(I) | 7.75 (1.40) | |
| Wilson B-factor | | 58.77 |
| R-merge | 0.1684 (1.324) | |
| R-means | 0.1898 (1.487) | |
| R-pim | 0.08488 (0.6573) | |
| CC½ | 0.994 (0.622) | |
| CC* | 0.998 (0.876) | |
| Reflections used in refinement | 49302 (4864) | |
| Reflections used for R-free | 2000 (197) | |
| R-work | 0.2061 (0.2982) | |
| R-free | 0.2890 (0.3944) | |
| CC (work) | 0.937 (0.757) | |
| CC (free) | 0.886 (0.616) | |
| Number of non-hydrogen atoms | | 12366 |
| macromolecules | | 12258 |
| ligans | | 108 |
| Protein residues | | 1452 |
| RMS (bonds) | | 0.111 |
| RMS (angles) | | 1.78 |
| Ramachandran favored (%) | | 92.96 |
| Ramachandran allowed (%) | | 6.97 |
| Ramachandran outliers (%) | | 0.07 |
| Rotamer outliers (%) | | 0.34 |
| Clashscore | | 14.19 |
| Average B-factor | | 60.62 |
| macromolecules | | 60.49 |
| ligands | | 74.8 |

The compound of Formula IV is constructed such that one end of its macrocycle is composed of a short, (D)Ser-(L)Phe-(L)Gly-(D)Arg stretch of residues (FIG. 2A). This portion of the compound orients the serine and arginine side chains atop a largely hydrophilic surface on GyrA (FIG. 14A). The phenylalanine on the macrocycle, as well as nearby methylated histidine, do not appear to directly contact GyrA in the structure. The opposite end of the compound is a branch terminating in a N-formylated tryptophan residue. Surprisingly, the indole ring of this tryptophan moiety occupies a pocket that has been previously shown to be exploited by the azaindole or chlorophenyl groups of thiophenes, a synthetic class of gyrase poisons that act by an allosteric mechanism (FIG. 14A to FIG. 14C).

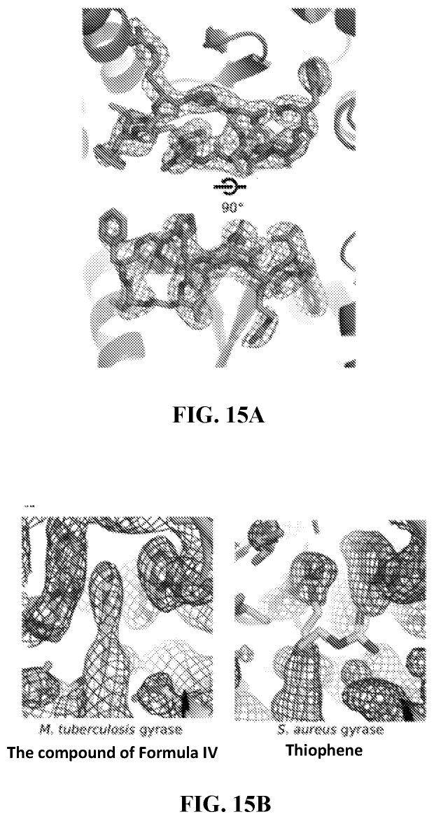

Given that resistance mutations obtained for the compound of Formula IV map to the region of the enzyme where fluoroquinolones bind, the placement observed for the compound was unexpected. However, inspection of the structure revealed a conformational commonality that is shared with a thiopene-inhibited complex obtained with the cleavage core of the S. aureus enzyme, but not with gyrases bound to fluoroquinolone poisons. In particular, the compound of Formula IV and thiopene-bound gyrase structures both contain an unusual architecture in which an arginine (R482 in MtbGyrase and R458 in S. aureus gyrase) intercalates between the DNA bases present in one of the enzyme's two cleavage centers, displacing one of the bases from that active site. This conformation is striking, given that the arginine sits >20 Å from the site of the compound of Formula IV or thiophene binding. The position of the intercalating arginine also overlaps with the site where a fluoroquinolone would normally bind to the enzyme (FIG. 14C, FIG. 15A and FIG. 15B).

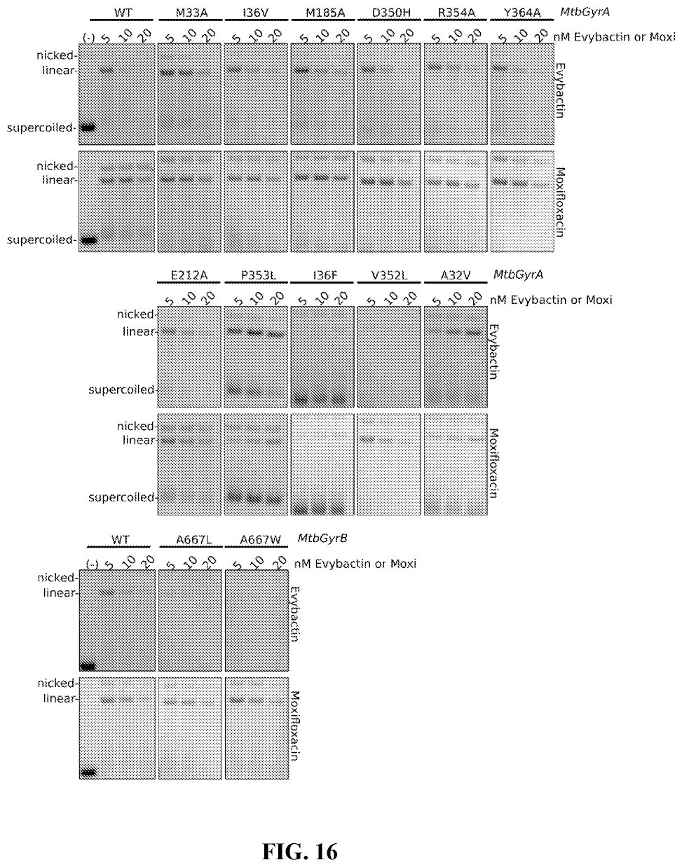

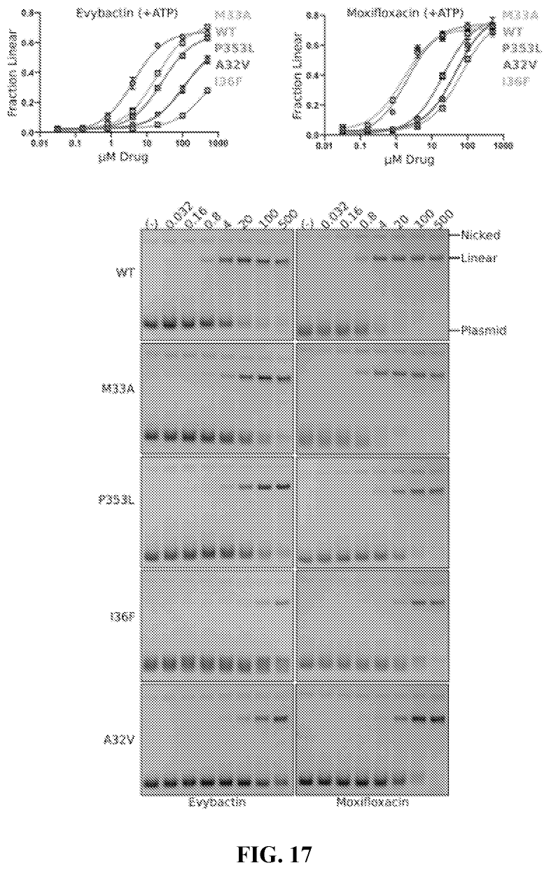

To further probe the nature of the compound of Formula IV-gyrase interaction, we generated 13 MtbGyrase variants that contain single mutations in the binding surface for the compound. The purified constructs were then screened to look for changes in the ability of the compound of Formula IV to promote DNA cleavage using an agarose gel-based cleavage assay. Consistent with the extended binding site of the compound of Formula IV along the surface of MtbGyrBA_{core}, which is mostly composed of weak van der Waals interactions (FIG. 14B), many variants had no or little effect on the compound of Formula IV-induced cleavage (FIG. 16). However, MtbGyrA_{M33A}, MtbGyrA_{P353L}, MtbGyrA_{A32V}, and MtbGyrA_{I36F} all displayed reduced cleavage (5 to 100-fold) in the presence of the compound of Formula IV compared to wildtype MtbGyrase (FIG. 17). These changes map to the hydrophobic binding pocket shared by the thiophenes and the tryptophan residue of the compound of Formula IV.

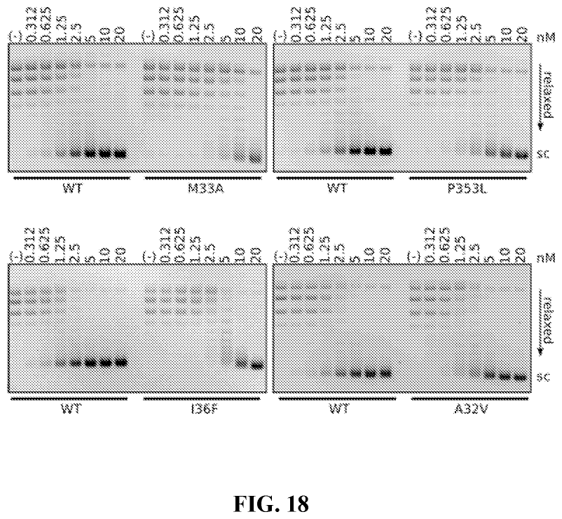

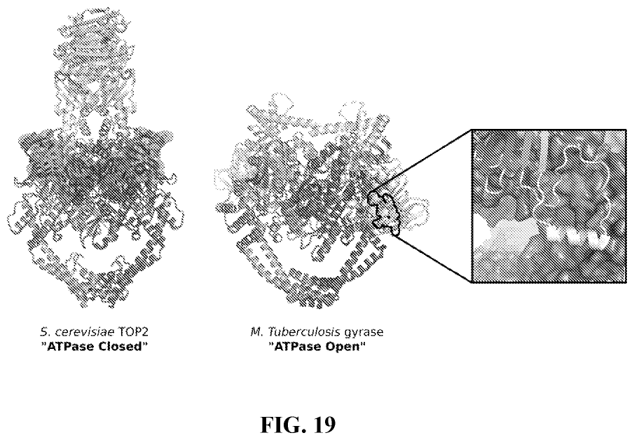

Interestingly, along with reduced sensitivity to the compound of Formula IV, both MtbGyrA_{M33A} and MtbGyrA_{I36F} also exhibited a 5 to 10 fold reduction in supercoiling activity by the mutant enzymes compared to wild type MtbGyrase (FIG. 18). This general loss of function may account for the reduced cleavage seen for these constructs in the presence of the compound of Formula IV. By comparison, the general supercoiling activity of MtbGyrA_{P353L} and MtbGyrA_{A32V} were only slightly reduced overall but showed relatively strong resistance against the compound of Formula IV (FIGS. 17 and 18). In E. coli, these mutations also give rise to resistance to thiophenes (P. F. Chan, et al. Thiophene antibacterials that allosterically stabilize DNA-cleavage complexes with DNA gyrase, Proc Natl Acad Sci USA 114, E4492-E4500 (2017)), consistent with our structural data showing that the two agents share a common binding pocket. Interestingly, in addition to reducing the cleavage-promoting efficiency of the compound of Formula IV, MtbGyrA_{P353L} and MtbGyrA_{A32V} also promoted resistance to cleavage induced by moxifloxacin (FIG. 17), despite the fact that these amino acid substitutions are far removed from the site of fluoroquinolone binding. This result indicates that there is allosteric coupling between the binding sites for the two classes of poisons.

A differential screen of a small collection of Photorhabdus symbionts of the nematode microbiome resulted in the isolation of the compound of Formula IV, a novel cyclic depsipeptide antibiotic acting potently and selectively against M. tuberculosis. The compound is highly polar, and not well suited to diffuse across a hydrophobic cytoplasmic membrane. The target is intracellular, the well-conserved bacterial DNA gyrase. All currently known compounds acting selectively against M. tuberculosis hit a unique target (or a unique site). This is not the case with the compound of Formula IV—its activity depends on the BacA transporter, which explains both the penetration of this polar compound into the cell, and the mechanism of selectivity. BacA is an unusual ABC-type “multisolute transporter” that apparently transports vitamin B12 into the cell. The same transporter was also found to translocate hydrophilic bacitracin into M. tuberculosis. Mycobacteria seem to be a rare group of Gram-positive species to harbor BacA: the only other example is Streptococcus pneumoniae which has a microcin B17 transporter with 99.7% identity to E. coli SbmA and was probably acquired via horizontal gene transfer; other members of this family of transporters are sparsely scattered among Gram-negative species. BacA-type transporters are absent in human gut symbionts, explaining low activity of the compound of Formula IV against them. Notably, the compound of Formula IV has low activity against wild type E. coli carrying the SbmA homolog of BacA but is very potent against a mutant with a disrupted outer membrane permeability barrier. Our results suggest that in E. coli, the compound of Formula IV penetration is restricted by the outer membrane and efflux by multidrug pumps, and only some of the compound gets smuggled into the cell by SbmA. We expect that the compound of Formula IV will be similarly inactive against other Gram-negative bacteria.

The mechanism of action for the compound of Formula IV is distinct from that of fluoroquinolones. The compound of Formula IV binds at an allosteric site distal from the site of fluoroquinolone binding. A portion of this locus was first identified as a binding pocket for a class of gyrase poisons known as thiophenes, a group of antagonists identified from unbiased high-throughput screens of synthetic compounds against E. coli gyrase. The existence of natural products that target this allosteric site highlights the importance of the pocket as a critical node for gyrase activity and a point for small molecule intervention. Interestingly, DNA cleavage induced by the compound of Formula IV is highly ATP dependent, further distinguishing this molecule from fluoroquinolones. Comparative structural analyses of the compound of Formula IV binding pocket reveals it is also the binding site for a corynebacterial-specific loop within the ATPase domains of DNA gyrase. In the absence of ATP and DNA, the ATPase domains of MtbGyrase fold down against the cleavage core of the enzyme, an action that we find occludes the compound of Formula IV-binding pocket (FIG. 18). This “open” conformation of the ATPase domains within MtbGyrase may account for the strict ATP dependence for the compound of Formula IV-induced cleavage.

Treating tuberculosis requires a constant introduction of novel compounds to combat emerging resistance. The rise of MDR and XDR-TB strains resistant to most currently available antibiotics underscores the need for new therapies. BacA null mutants resistant to the compound of Formula IV occur with high frequency but have reduced virulence. This correlation suggests that only low-frequency gyrase mutants may pose a problem. In the case of M. tuberculosis, this concern is ameliorated, since all drugs are introduced as combinations. So far, each new compound has been added to an existing regimen, which provides only a temporary relief from emerging resistance. Ideally, such combinations should only contain novel compounds free of preexisting resistance; this approach will effectively prevent resistance development. Drug combinations should also consist of M. tuberculosis-selective compounds to avoid harming the microbiome. The current pipeline of anti-TB drugs in development and efficient methods to discover novel selectively acting natural products make this strategy realistic.

### Examples

The following examples illustrates the invention without limiting its scope. Synthesis of the Compound Represented by Formula IV.

### Screening Condition

Photorhabdus sp. and Xenorhabdus sp. were cultivated in LBB, TSB and Nutrient Broth (NB) for 8 days at 28° C. Concentrated culture extract and S. aureus HG003 overlay plate for antibacterial assay were prepared as previously. For screening against M. tuberculosis was performed as below. M. tuberculosis H37Rv mc²6020 (ΔlysA ΔpanCD) expressing mCherry (ΔlysA ΔpanCD, pBEN_mCherry kan^{r}) was cultured in supplemented Difco 7H9 medium containing kanamycin and incubated at 37° C. and 100 rpm. The culture was diluted into fresh medium to a final OD₆₀₀ of 0.003 and 148.5 μL of culture was added to the wells of the 96-well black with flat, clear bottom microtiter plate (Corning) containing the 1.5 μl culture extract (1:100 dilution). The plate was incubated for 7 days at 37° C. and 100 rpm, at which point the optical density at 600 nm and fluorescence with excitation at 580 nm and emission at 610 nm were measured on a plate reader. The extract was deemed to have activity against M. tuberculosis H37Rv mc²6020 as it had ≥75% growth inhibition when compared to the growth control. The assay was repeated for confirmation of activity. The culture samples which only showed activity against M. tuberculosis H37Rv mc²6020 were determined as anti-TB selective extract.

### Purification of the Compound of Formula IV

P. noenieputensis DSM 25462 was inoculated in 500-ml Erlenmeyer flask with 200 ml LB broth and incubated at 28° C. with aeration at 200 rpm for over-night. Ten milliliter of over-night culture was inoculated into a 2-1 Erlenmeyer flask with 1 L TNM-FH insect medium (Sigma-Aldrich) and incubated for 10-14 days. Cell were removed by centrifugation (8,000×g, 5 min), and supernatant was incubated with XAD16N resin (20-60 mesh, Sigma-Aldrich) for over-night. After removed supernatant, the compound represented by Formula IV was eluted from XAD16N resin by 100% methanol. Samples were dried and resuspended in 5 ml MilliQ water. 5 ml concentrated culture extract was subjected to reverse-phase HPLC on a C18 column (Luna® 5 μm C18(2) 100 Å, LC Column 250×21.2 mm, Phenomenex). HPLC conditions were as follows: solvent A, Milli-Q water and 0.1% (v/v) formic acid; solvent B, acetonitrile and 0.1% (v/v) formic acid. The initial concentration of 10% solvent B was maintained for 5 min, followed by a linear gradient to 35% over 20 min, and maintained at 100% for 10 min with a flow rate of 7 ml min⁻¹; fractions were collected every 20 second, UV detection by diode-array detector from 210 to 400 nm, and active fraction was eluted 19-24 min. Active fraction was subjected to reverse-phase HPLC on a C18 column (XBridge®, BEH C18 OBD prep column, 100 Å, 5 μm: 250 mm×10 mm, Waters). HPLC conditions were as follows: solvent A, Milli-Q water and 0.1% (v/v) formic acid; solvent B, acetonitrile and 0.1% (v/v) formic acid. The initial concentration of 10% solvent B was maintained for 2 min, followed by a linear gradient to 38% over 8 min with a flow rate of 5 ml min⁻¹; UV detection by diode-array detector from 210 nm. The compound of Formula IV was eluted at 8 min, with a purity of 92% by UV.

### Structure Elucidation

LC-MS analysis was conducted on a 6530 Q-TOF-LC/MS (Agilent Technologies, Palo Alto, CA, USA). The HPLC column was a reversed-phase ZORBAX RRHT Extend-C18, 2.1×50 mm, 1.8 μm (Agilent Technologies, USA). The mobile phases were water with 0.1% formic acid (A) and acetonitrile with 0.1% formic acid (B). A linear gradient was initiated with 2% acetonitrile and linearly increased to 52% at 2-12 min. The flow rate was 0.2 ml min, and the injection volume was 5 μl. Mass spectra in the m/z range 111-3000 were obtained by positive ion (+ESI) modes. The mass spectrometry conditions were as follows: gas temperature 300° C., N₂ flow rate 7 L min⁻, nebulizer gas pressure 35 psig, capillary voltage 3500 V, fragmentor potentials 175 V, Vcap 3500 V, Skimmer 65 V, and Octopole RFPeak 750 V. Data acquisition and analysis were conducted using Agilent LC-MS-QTOF MassHunter Data Acquisition Software version 10.1 and Agilent MassHunter Qualitative Analysis Software version 10.0, respectively (Agilent Technologies, USA).

All NMR data were recorded on a Bruker AVANCE II 700-MHz spectrometer with 5 mm TXI probehead, and a 600-MHz spectrometer with a cryoprobe. All NMR experiments were performed with 10 mg of the compound of Formula IV spiked with tetramethylsilane (TMS) in DMSO-d₆ (320K) and 4% D₂O in H₂O (300K). All carbon and proton chemical shifts were referenced by TMS. Complete assignments were obtained using two-dimensional experiments, including COSY (cosygpmfqf), TOCSY (dipsi2etgpsi), ¹H-¹³C HSQC (hsqcedetgpsisp2.3), ¹H-¹⁵N HSQC (hsqcetf3gpsi2), ¹H-¹³C HMBC (hmbcgplpndqf), ROESY (roesyphpp.2) and 1,1-ADEQUATE (adeq1 letgprdsp_bbhd).

### MIC and Cytotoxicity

M. tuberculosis strains including strain H37Rv mc²6020 (ΔlysA ΔpanCD), H37Rv mc²6020 expressing an mCherry plasmid and conferring kanamycin resistance (ΔlysA ΔpanCD, pBEN_mCherry kan^{r}) and the compound of Formula IV resistant mutants of H37Rv mc²6020, and Mycobacterium smegmatis mc²155 and Mycobacterium abscessus ATCC 19977 were used in this study. The MIC was determined for M. tuberculosis H37Rv mc²6020, H37Rv mc²6020 expressing mCherry, M. smegmatis, and M. abscessus by broth microdilution. For all strains, a final OD₆₀₀ of 0.003 was obtained by diluting an exponentially growing culture of bacteria into supplemented 7H9 medium (10% Middlebrook Oleic Albumin Dextrose Catalase (OADC) Growth Supplement form (Millipore Sigma), 5% glycerol, 1% casamino acids, 0.05% tyloxapol, 80 μg ml⁻¹ lysine, 24 μg ml⁻¹ pantothenate, and 50 μg ml⁻¹ kanamycin where appropriate for M. tuberculosis strains, 10% ADC, 5% glycerol, and 0.05% tyloxapol for non-tuberculosis strains). The plates were incubated 37° C. and 100 rpm for 7 days (M. tuberculosis) or 3 days (M. smegmatis, M. abscessus). The MIC was defined as the lowest concentration of antibiotic with no visible growth. MIC of the compound of Formula IV against aerobic and anerobic bacteria, human commensal bacteria and cytotoxicity assay were performed as previously.

### Time-Dependent Killing

The exponential cultures of M. tuberculosis H37Rv mc²6020 were prepared by growing the strain to mid-exponential (OD₆₀₀ 1-1.5) and then backdiluting to OD₆₀₀ 0.003. For stationary phase cultures, M. tuberculosis H37Rv mc²6020 was grown for two weeks to an OD₆₀₀>1.5. Cultures were challenged with either 10×MIC of the compound of Formula IV or moxifloxacin. Cultures were incubated at 37° C. and 100 rpm. At intervals, 100 μL aliquots were removed from each culture, serially diluted, and plated onto supplemented 7H10 medium to determine CFU per ml. The exponential phase plates were incubated for three weeks and the stationary phase plates were incubated for two weeks prior to counting. both at 37° C. Experiments were performed with biological and technical replicates.

Staining and Fluorescent Imaging of M. tuberculosis

M. tuberculosis cultures were grown to exponential phase (OD₆₀₀-0.5) then treated with 10×MIC of the indicated antibiotic. Aliquots were taken after 48 hours of treatment, washed once in PBS+0.05% Tween-80 (PBST), and fixed in 4% paraformaldehyde for 2 hours at room temperature. The cells were washed once, resuspended in PBST and stained with FM4-64FX (Thermofisher) at a final concentration of 1 μg ml⁻¹ for 30 minutes in the dark at room temperature. Once stained 1 μl of cells were spotted onto a 1.5% low-melting agarose pad and observed with a Nikon Ti2-E inverted fluorescence microscope using a 100× oil-immersion objective lens. Images were acquired by NIS-Elements at a resolution of 2,048×2,048 and processed with Fiji software. Segmentation and calculation of cellular length from these images was done using the plug-in MicrobeJ.

### Resistant Study

Mutants to the compound of Formula IV in M. tuberculosis H37Rv mc²6020 were selected by plating on supplemented 7H10 medium containing 10×, and 100×MIC of the compound of Formula IV. Isogenic cultures of M. tuberculosis H37Rv mc²6020 were obtained by plating 100 μl of an exponentially growing culture onto supplemented 7H10 medium without antibiotics and incubating at 37° C. for three weeks. Three independent colonies were picked and inoculated into 10 ml supplemented 7H9 medium, grown for two weeks, subcultured 1:100 into 40 ml of supplemented 7H9 medium, and grown to an OD₆₀₀-1.0. Cells were plated at 107, 108, and 109 concentrations, this was achieved by: removing 100 μl of culture, serially diluted, and plated in triplicate for CFU; removing 4400 μl of culture, plating 400 μl (100 μl per plate) onto plates containing either 10×MIC of the compound of Formula IV and rifampicin, the remaining 4 ml was pelleted by centrifugation at 8000 rpm for 5 minutes, resuspended in 400 μl supplemented 7H9 medium, and plated as previously described: the remaining 36 ml was washed once, pelleted by centrifugation at 8000 rpm for 5 minutes, resuspended in 4 ml supplemented 7H9 medium, and 200 μl was plated onto each of the remaining plates, 5 for each antibiotic. This was repeated for each isogenic culture at 100×MIC. The plates were incubated at 37° C. for three weeks, at which point the number of colonies on each plate were counted. Mutation frequency was calculated by dividing the number of mutants obtained by the total bacteria plated. The mutants were picked and inoculated into 10 ml supplemented 7H9 medium without antibiotics, grown for two weeks, and subcultured 1:100 into 10 ml supplemented 7H9 medium without antibiotics. The broth MIC for the mutants was determined to confirm maintenance of the compound of Formula IV resistance. Genome sequencing and variant calling were conducted by Microbial Genome Sequencing Center (MiGS; Pittsburgh, PA). Whole genome sequence was performed by pair end reads (2×150 bp) with Illumina NextSeq 550, and M. tuberculosis genome information data in NCBI (NCBI Reference Sequence: NC_000962.3) was used for variant calling.

Targeted Mutation of bacA and gyrA

Targeted deletion of bacA in M. tuberculosis H37Rv mc²6020 was accomplished as in previous study. The plasmid pMSGzeo was used to construct the recombination substrate consisting of a zeocin resistance marker flanked by DNA fragments with homology to the upstream and downstream regions of bacA (D. Barkan, et al., Redundant function of cmaA2 and mmaA2 in Mycobacterium tuberculosis cis cyclopropanation of oxygenated mycolates. J Bacteriol 192, 3661-3668 (2010)). The sequence of the primers used to amplify the flanking regions are: bacAKOLFF or—5′-TTAAGATCTCGGGCCACCGGCGCCACAAAC-3′ (SEQ ID NO. 1), bacAKOLFRey—5′-GGGAAGCTTAAACAATTTCGGGCCCAAGG-3′ (SEQ ID NO. 2), bacAKORFF or—5′-GGGTCTAGAACGCTGAATCCGTCGATCTC-3′ (SEQ ID NO. 3), bacAKORFRev—5′-TTTGGTACCCTCCGTTACCGATCAGTGG-3″ (SEQ ID NO. 4). Null mutants were selected on 7H10 agar supplemented with 100 μg ml⁻¹ zeocin. Mutation was confirmed via PCR and sequencing. Point mutations in M. tuberculosis gyrA were constructed via single stranded recombineering as in previous study (J. C. van Kessel, et al., Efficient point mutagenesis in mycobacteria using single-stranded DNA recombineering: characterization of antimycobacterial drug targets. Mol Microbiol 67, 1094-1107 (2008)) with plating on 100× MIC the compound of Formula IV. Sequence of oligonucleotides used to make targeted mutations were gyrA G88C—5′-ATGCGCACCAGGCTGTCGTAGATCGACGCGTCGCAGTGC GGGTGGTAGTTGCCCATGGTCTCGGCAACCG-3′ (SEQ ID NO. 5), gyrA G88S—5′-ATGCGCACCAGGCTGTCGTAGATCGACG CGTCGCTGTGCGGGTGGTAGTTGCCCATGGTCTCGGCAACCG-3′ (SEQ ID NO. 6).

Targeted mutations were confirmed via PCR and sanger sequencing.

### Macromolecule Incorporation Assay

Macromolecular synthesis assay against DNA, RNA, protein and fatty acid were analyzed by ImQuest BioSciences Inc. (MD, USA) with E. coli WO153 cells.

### Purification of Recombinant MtbGyrase

Full length M. tuberculosis GyrA and GyrB were prepared as previously described (T. R. Blower, et al., Crystal structure and stability of gyrase-fluoroquinolone cleaved complexes from Mycobacterium tuberculosis. Proc Natl Acad Sci USA 113, 1706-1713 (2016)). Briefly, M. tuberculosis GyrA and GyrB were expressed separately from modified pET vectors containing an N-terminal hexahistidine SUMO tag, using BL21[DE3] CodonPlus E. coli cells (Agilent). Cells were grown at 37° C. to mid log phase in 2× TY media, after which the temperature was reduced to 30° C. and protein production induced with 0.5 mM IPTG for 4 hours before harvesting by centrifugation. Cells were resuspended in A1000 (30 mM Tris-HCl (pH 7.8): 1 M NaCl: 10 mM imidazole, pH 8.0; 10% glycerol: 0.5 mM TCEP: 1 μg ml⁻¹ leupeptin: 1 μg ml⁻¹ pepstatin: 1 mM PMSF). GyrA and GyrB were purified separately, following an identical procedure. Cells were lysed by addition of 1 mg ml-1 egg white lysozyme, followed by sonication. Cell lysate was then clarified by centrifugation and the soluble lysate applied to a 5 ml HisTrap-HP column (Cytiva). The column was washed with 200 ml A1000, followed by elution with 30 ml B1000 Å1000 (30 mM Tris-HCl (pH 7.8): 1 M NaCl: 500 mM imidazole, pH 8.0; 10% glycerol: 0.5 mM TCEP: 1 μg ml⁻¹ leupeptin: 1 μg ml⁻¹ pepstatin: 1 mM PMSF). SUMO tagged protein was then cleaved with SENP protease and dialyzed overnight against A500 (30 mM Tris-HCl (pH 7.8): 500 mM NaCl: 500 mM imidazole, pH 8.0; 10% glycerol: 0.5 mM TCEP: 1 μg ml⁻¹ leupeptin: 1 μg ml⁻¹ pepstatin: 1 mM PMSF). Cleaved protein was passed over a 5 ml HisTrap-HP column and the flow-through then collected and concentrated. GyrA and GyrB were each run separately over a Superdex 200 10/300 column (Cytiva) equilibrated in C500 (50 mM Tris-HCl (pH 7.8): 500 mM KCl: 10% glycerol: 0.5 mM TCEP). Peak fractions were collected, concentrated and the final glycerol concentration increased to 30% before flash freezing in liquid nitrogen for storage at −80° C.

### Supercoiling and Supercoil Relaxation Assays

Purified M. tuberculosis GyrA and GyrB were mixed on ice in an equimolar ratio to form gyrase heterotetramers at a concentration of 40 μM. MtbGyrase was then serially diluted in two-fold steps using gyrase dilution buffer [50 mM Tris-HCl (pH 7.8): 150 mM monopotassium glutamate: 5 mM magnesium acetate: 10% glycerol] to 10× working concentrations for supercoiling assays. Supercoiling assays were assembled by mixing the following on ice: 2 μl 10× relaxed pSG483 plasmid DNA (68.75 nM), 2 μl 10× gyrase dilutions (3.12 nM-200 nM), 7 μl deionized water, 4 μl 5× reaction buffer [120 mM Tris-HCl (pH 7.8): 38 mM magnesium acetate: 340 mM monopotassium glutamate: 36% glycerol: 0.4 mg ml⁻¹ BSA: 4 mM TCEP], and 2 μl deionized water or 10× the compound of Formula IV (1 mM). Reactions were initiated by the addition of 2 μl 10×ATP (20 mM) and then incubated at 37° C. for 30 minutes before quenching using 3 μl reaction stop buffer [125 mM EDTA pH 8.0; 5% SDS], followed by addition of 2 μl of 3 mg ml⁻¹ proteinase K. Reactions were digested of protein by further incubation at 37° C. for 30 minutes. Loading dye (5 μl of 5× loading dye) was added to reactions and products were resolved on a 1.5% native TAE agarose gel by running at 35 V for 16.5 hours. Gels were post-stained with ethidium bromide and visualized by UV transillumination.

### ATPase Assays

ATPase assays were conducted using an NADH-coupled assay. Gyrase heterotetramer was formed as described above for the supercoil relaxation assays. Reactions were assembled in the following manner on ice: 5 μl 10× gyrase (2.5 μM), 5 μl 10× sheared salmon sperm DNA (2.5 mg ml⁻¹), 5 μl deionized water, 25 μl 2× Buffer/NADH solution (100 mM Tris pH 7.8; 170 mM monopotassium glutamate: 10% glycerol: 0.2 mg ml⁻¹ BSA: 5 mM magnesium acetate: 7 mM phosphoenolpyruvate: 0.6 mM NADH: 10% Pyruvate Kinase/Lactic Dehydrogenase enzymes from rabbit muscle (Sigma-Aldrich)), and 5 μl 10× the compound of Formula IV or moxifloxacin (1 mM). Samples were incubated at 37° C. before the addition of 5 μl 10×ATP (0.625 mM to 40 mM) to initiate the reactions. Absorbance changes at 340 nm were observed over 30 minutes and absorbance change over time corresponding to ATP consumption was calculated using NADH standard curves. K_{cat}, K_{M} and Vₘₐₓ values were calculated by nonlinear curve fitting in Prism Graphpad 8 using the Mechaelis-Menten equation.

### Plasmid Cleavage Assays

Plasmid cleavage assays were conducted similarly to the supercoiling and supercoil relaxation assays, with a few modifications. Gyrase stock concentrations for the cleavage assays were 200 nM (final concentration 20 nM) and the compound of Formula IV or moxifloxacin stock concentrations were 64 nM to 1 mM (final concentrations 6.4 nM to 100 μM). Cleavage assays were resolved on a 1.5% TAE agarose gel containing 1 μg ml⁻¹ ethidium bromide by running at 35 V for 16.5 hours. Gels were then post-stained with ethidium bromide and visualized by UV transillumination. ImageJ was used for quantitation of cleaved products and fraction plasmid cleaved was calculated taking the cleaved band intensities and dividing by the sum of the cleaved band and supercoiled band intensities. IC₅₀ values were calculated by nonlinear curve fitting using Prism Graphpad 8 using the following equation−Y=Min+X*(Max-Min)/(IC50+X).

### MtbGyrBA_{core}(Y129F) Purification and X-Ray Crystallography

MtbGyrBA_{core}(Y129F) was purified as previously described (T. R. Blower, et al., Crystal structure and stability of gyrase-fluoroquinolone cleaved complexes from Mycobacterium tuberculosis, Proc Natl Acad Sci USA 113, 1706-1713, (2016)). Briefly, the protein was expressed from a modified pET vector containing an N-terminal hexahistidine SUMO tag, using BL21[DE3] CodonPlus E. coli cells (Agilent). Cells were grown at 37° C. to OD₆₀₀ of 0.4 in M9ZB media (F. W. Studier, Protein production by auto-induction in high-density shaking cultures, Protein Expres Purif 41, 207-234, (2005)), after which the temperature was reduced to 18° C. and protein production induced with 0.25 mM IPTG for 18 hours before harvesting by centrifugation. Cells were resuspended in A1000 buffer, lysed, clarified and captured on a 5 ml HisTrap-HP column as described above. Captured protein was washed on the column with 50 ml A100 [30 mM Tris-HCl (pH 7.8): 100 mM NaCl: 10 mM imidazole, pH 8.0; 10% glycerol: 0.5 mM TCEP: 1 μg ml⁻¹ leupeptin: 1 μg ml⁻¹ pepstatin: 1 mM PMSF] to reduce the salt before eluting directly onto a 5 ml HiTrapQ-HP column (Cytiva) using B100 [30 mM Tris-HCl (pH 7.8): 100 mM NaCl: 10 mM imidazole, pH 8.0; 10% glycerol: 0.5 mM TCEP: 1 μg ml⁻¹ leupeptin: 1 μg ml⁻¹ pepstatin: 1 mM PMSF]. The HiTrapQ-HP column was washed with 5 column volumes of Q100 [30 mM Tris-HCl (pH 7.8): 100 mM NaCl: 10% glycerol: 0.5 mM TCEP: 1 μg ml⁻¹ leupeptin: 1 μg ml⁻¹ pepstatin: 1 mM PMSF] and a gradient of 0) to 100% Q1000 [30 mM Tris-HCl, pH 7.8; 1 M NaCl: 10% glycerol: 0.5 mM TCEP: 1 μg ml⁻¹ leupeptin: 1 μg ml⁻¹ pepstatin: 1 mM PMSF] over 10 column volumes was conducted to elute captured protein. Peak fractions were pooled and the hexahistidine-SUMO tag removed through overnight cleavage with SENP protease. Cleaved protein was then applied to a 5 ml HisTrap-HP column, and the flow-through collected and concentrated. Subsequently, MtbGyrBA_{core}(Y129F) was then applied to a Superdex 200 16/60 column equilibrated in C₅₀₀, after which peak fractions were pooled and concentrated, and the final glycerol concentration increased to 30% before flash freezing in liquid nitrogen for storage at −80° C.

For co-crystallization, a DNA substrate was adapted from previous structural studies of S. aureus gyrase (B. D. Bax, et al. Type IIA topoisomerase inhibition by a new class of antibacterial agents, Nature 466, 935-951, (2010)) and designed to contain a single nick that is offset 2 nt from the center of the substrate as well the DNA ends joined by “GAA” triloop linkers: 5′-GGCCCTACGGCTgaaAGCCGTAGGGCCCTACGGCTgaaAGCCGTAG-3′ (SEQ ID NO. 7): The 2 nt offset positions the nick in one catalytic center of the enzyme to ensure a precise binding register with the protein. The oligo was ordered from IDT (Integrated DNA technologies) and annealed in [what] using a thermocycler to generate the appropriate substrate for crystallography. MtbGyrBA_{core(Y129F)} and annealed DNA were mixed in a 1:1.7 protein:DNA ratio (150 μM MtbGyrBA_{core(Y129F)} dimer; 255 μM DNA oligo). Protein:DNA complex was then dialyzed against 20 mM Tris-HCl (7.8): 150 mM NaCl 10 mM MgCl₂: 0.5 mM TCEP. Dialyzed protein:DNA complex was incubated with 1 mM the compound of Formula IV at 37° C. for 3 hours before conducting crystallization trials. Long, rod-like crystals formed after several days in hanging drops containing 7-12% PEG10K: 100 mM MES pH 6.0; 200 mM magnesium acetate. Crystals were cryopreserved in 12% PEG10K; 100 mM MES pH 6.0; 200 mM magnesium acetate: 1 mM TCEP: 1 mM the compound of Formula IV: 25% glycerol. Diffraction data were collected at NSLS-II beamline 17-ID-2 (FMX) and initially autoprocessed using Fast DP (G. Winter, et al., Automated data collection for macromolecular crystallography. Methods 55, 81-93 (2011)). Further data processing and data reduction was carried out using XDS and CCP4. Molecular replacement was conducted using Phaser (Mccoy, A. J. et al. Phaser crystallographic software. J Appl Crystallogr 40, 658-674 (2007)) and a single monomer subunit of a prior MtbGyrBA_{core} model (PDB ID 5BTA), stripped of ligands, DNA and waters. Coot (P. Emsley, et al., model-building tools for molecular graphics, Acta Crystallogr D 60, 2126-2132 (2004)) was used for model building and ligand placement while refinement was conducted in Phenix and figures were generated using Pymol (The PyMOL Molecular Graphics System, Version 2.4 Schrödinger, LLC.)

### Animal Study

Animal study was performed at Northeastern University, approved by Northeastern IACUC, and was performed according to institutional animal care and policies. Experiments were not randomized nor blinded, as it was not deemed necessary. Female CD-1 mice (20-25 g, experimentally naive, 6 weeks old) from Charles River were used for all studies. The compound of Formula IV was tested in a septicemia model against E. coli ATCC 25922. Mice were infected with 0.5 ml of E. coli ATCC 25922 suspension in BHI with 5% mucin (1×10⁶ to 5×10⁶) via intraperitoneal injection. This dose achieves >83% mortality within 24 h after infection. At 1 h after infection, mice were treated by the compound of Formula IV from 10 mg kg⁻¹ to 100 mg kg⁻¹ by intraperitoneal injection. Infection control mice were treated with 50 mg kg⁻¹ gentamicin as positive control. Survival was monitored for 120 h.

1. A compound of Formula I, 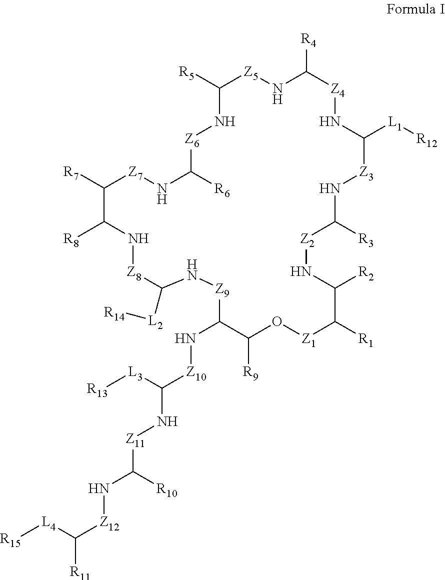
or a pharmaceutically acceptable salt, solvate or stereoisomer thereof:
wherein, in Formula I,
R₁ to R₁₀ are each independently selected from the group consisting of hydrogen, alkyl, alkenyl, alkynyl, hydroxyl, hydroxyalkyl, halogen, —CN, —O-alkyl, —C(O)-alkyl, —C(O)O-alkyl, —C(O)OH, —C(O)NH₂, —C(O)NH-alkyl, —NH₂, —NO₂, —CF₃, —NH-alkyl, —N-(alkyl)₂, —NHC(O)-alkyl, aryl, alkylaryl, alkylheteroaryl, wherein said alkyl, alkenyl, alkynyl and aryl are each optionally substituted;
R₁₁, R₁₂ and R₁₃ are each independently selected from the group consisting of hydrogen, alkyl, alkenyl, alkynyl, hydroxyl, hydroxyalkyl, halogen, amine, —NHC(NH)NH₂, —NHC(O)NH₂, —NHC(O)CH₃, —NHSO₂NH₂, —NHSO₂CH₃, —NHSO₂C₆H₅, —NHCHO wherein said alkyl, alkenyl, alkynyl and aryl are each optionally substituted;
R₁₄ is selected from the group consisting of imidazole, pyrazole, triazole, oxazole, isoxazole, thiazole, isothiazole, oxadiazole, thiadiazole and tetrazole, wherein each member of the group is optionally substituted;
R₁₅ is selected from the group consisting of indole, benzothiophene, benzoxazole, benzofuran, benzothiazole, benzimidazole, benzoxadiazole, benzothiadiazole, benzotriazole, pyrazolopyridine, imidazopyridine, pyrrolopyridine, pyrrolopyrimidine, indolizine, and purine, wherein each member of the group is optionally substituted;
L₁ to L₄ are each independently a bond or —(CH₂)ₙ—, wherein n is an integer between 0 and 10; and
Z₁ to Z₁₂ are each independently selected from the group consisting of —C(O)—, —CH₂—, —C(OH)—, —C(O)O-alkyl, and —C((O)alkyl)-.

2. The compound according to claim 1, wherein the compound of Formula I has the Formula II: 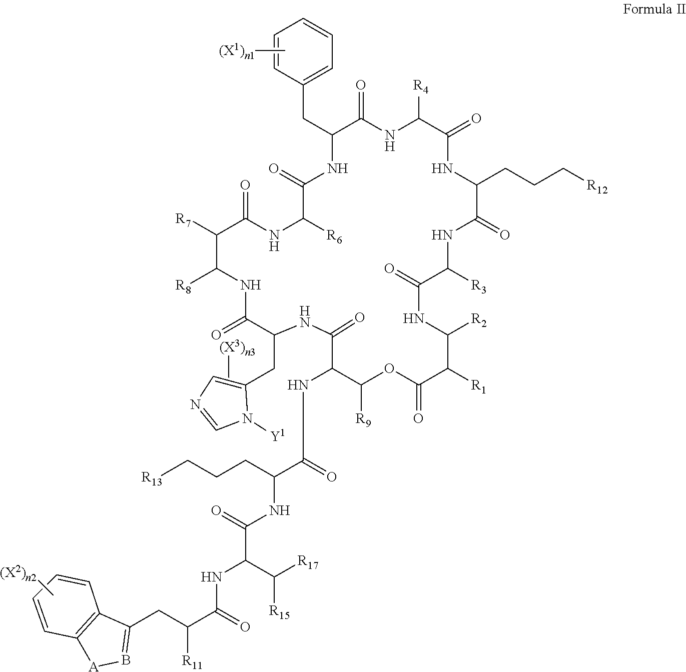
or a pharmaceutically acceptable salt, solvate or stereoisomer thereof:
wherein R₁ to R₄ and R₆ to R₁₃ are as defined in claim 1;
R₁₆ and R₁₇ are each independently selected from the group consisting of hydrogen, alkyl, alkenyl, alkynyl, hydroxyl, hydroxyalkyl, halogen, —CN, —O-alkyl, —C(O)-alkyl, —C(O)O-alkyl, —C(O)OH, —C(O)NH₂, —C(O)NH-alkyl, —NH₂, —NO₂, —CF₃, —NH-alkyl, —N-(alkyl)₂, —NHC(O)-alkyl, -aryl, -alkylaryl, alkylheteroaryl, wherein said alkyl, alkenyl, alkynyl and aryl are each optionally substituted;
X¹ to X³ are each independently selected from the group consisting of halogen, hydroxyl, cyano, isocyano, nitro, amino, sulfanyl, carboxyaldehyde, hydroxycarbonyl, alkyl, haloalkyl, cyanoalkyl, and alkyloxy;
n1 to n3 are each independently an integer of 0 to 2;
Y₁ is selected from the group consisting of halogen, cyano, nitro, alkyl, alkoxy, alkylsulfanyl, alkyl substituted by halogen, —C(O)-alkyl, —C(O)—O-alkyl, and —NH—C(O)—O-alkyl; and
A and B are each independently N or CR₁₈, wherein R₁₈ is selected from the group consisting of hydrogen, optionally substituted alkyl, optionally substituted alkenyl, optionally substituted alkynyl, and optionally substituted cycloalkyl.

3. The compound according to claim 1 or 2, wherein R₁, R₄ and R₇ are hydrogen.

4. The compound according to any one of claim 1-3, wherein R₂ and R₈ are —C(O)OH.

5. The compound according to any one of claim 1-4, wherein R₃ and R₆ are —CH₂OH.

6. The compound according to any one of claim 1-5, wherein R₉ is —CH₃.

7. The compound according to any one of claim 1-6, wherein R₁₀ is —CH(OH)CH₃.

8. The compound according to any one of claim 1-7, wherein R₁₁ is —NHCHO.

9. The compound according to any one of claim 1-8, wherein R₁₂ and R₁₃ are —NHC(NH)NH₂.

10. The compound according to claim 1 or 2, wherein the compound is represented by Formula III: 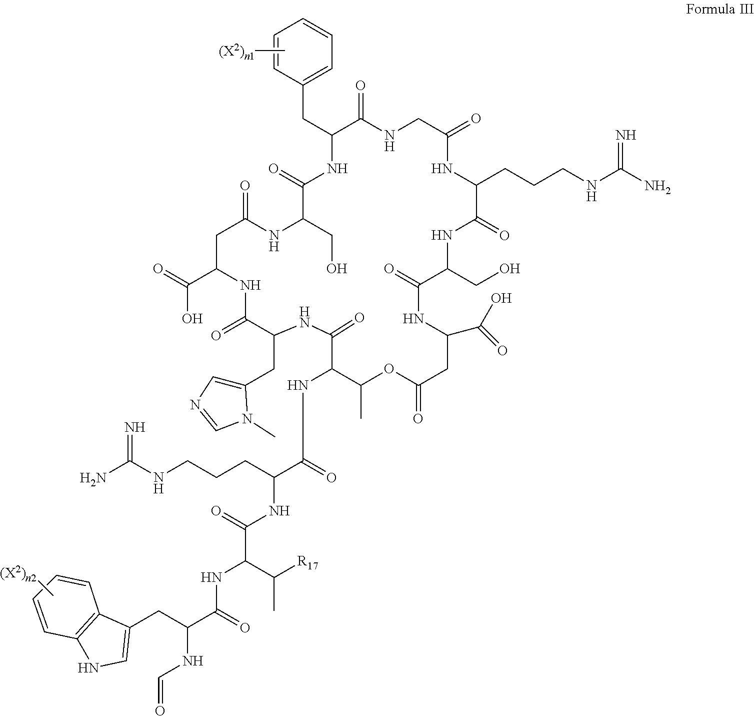
or a pharmaceutically acceptable salt, solvate or stereoisomer thereof:
wherein R₁₇, X¹, X², n1 and n2 are as defined in claim 2.

11. The compound according to claim 1 or 2, wherein the compound is represented by Formula IV: 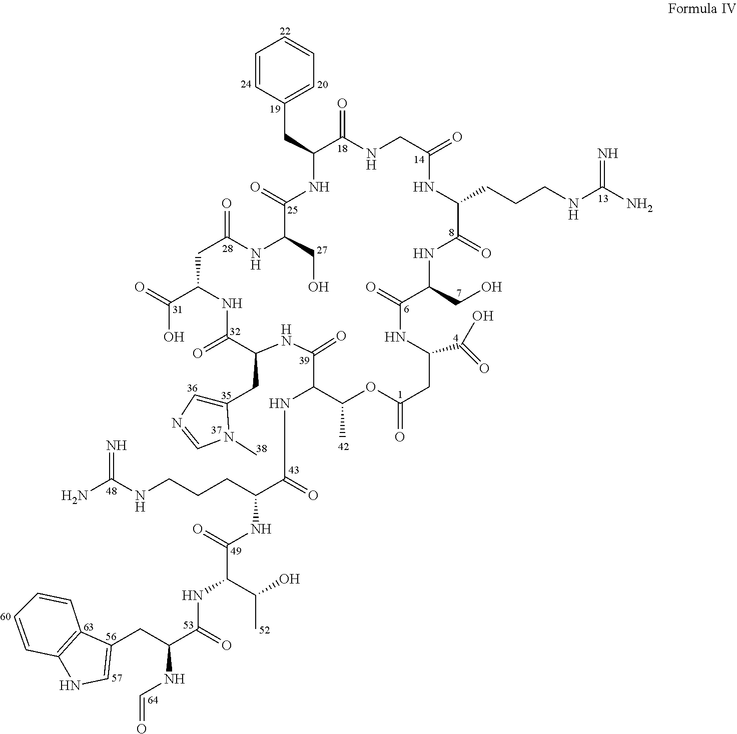 [SMILES: C[C@H]1OC(=O)C[C@@H](C(=O)O)NC(=O)[C@H](CO)NC(=O)[C@@H](CCCNC(=N)N)NC(=O)CNC(=O)[C@H](Cc2ccccc2)NC(=O)[C@@H](CO)NC(=O)C[C@@H](C(=O)O)NC(=O)[C@H](Cc2cncn2C)NC(=O)C1NC(=O)[C@@H](CCCNC(=N)N)NC(=O)[C@@H](NC(=O)[C@H](Cc1c[nH]c2ccccc12)NC=O)[C@@H](C)O]
or a pharmaceutically acceptable salt, solvate or stereoisomer thereof.

12. The compound according to any one of claim 1-11, wherein the compound is isolated in a stereochemically pure form from a microorganism.

13. A pharmaceutical composition for treating an infection caused by mycobacterium in a subject comprising a therapeutically effective amount of the compound according to any one of claim 1-12 or a pharmaceutically acceptable salt, solvate or stereoisomer thereof.

14. The pharmaceutical composition according to claim 13, further comprising at least one pharmaceutically acceptable carrier, excipient or diluent.

15. The pharmaceutical composition according to claim 13 or 14, in a form of topical administration, systemic administration, parenteral administration, subcutaneous administration, or transdermal administration, rectal administration, oral administration, intravaginal administration, intranasal administration, intrabronchial administration, intraocular administration, intra-aural administration, intravenous administration, intramuscular administration, or intraperitoneal administration.

16. The pharmaceutical composition according to any one of claim 13 to 15, further comprising at least one additional therapeutic agent.

17. The pharmaceutical composition according to any of claim 13 to 16, obtained by culturing a microorganism having an ability to produce the compound in a nutrient medium.

18. The pharmaceutical composition according to claim 17, wherein the microorganism is Photorhabdus noenieputensis DSM 25462.

19. A method of treating a disease or an infection caused by a bacterium in a subject in need thereof, comprising administering a therapeutically effective amount of one or more of the compounds according to any one of the claim 1 to 12, or pharmaceutically acceptable salts thereof, solvate or stereoisomer thereof.

20. The method of claim 19, wherein the compounds or pharmaceutically acceptable salts thereof, solvate or stereoisomer thereof are administered as a pharmaceutical composition comprising the compounds or pharmaceutically acceptable salts, solvates or stereoisomers thereof and a pharmaceutically acceptable carrier.

21. The method of claim 19 or 20, wherein the infection is a respiratory infection, a skin or skin structure infection, a urinary infection, an intra-abdominal infection, a blood stream infection, or a gastrointestinal infection.

22. The method of claim 19 or 20, wherein the infection is a Mycobacterium tuberculosis infection.

23. The method of claim 19 or 20, wherein the bacterium is a Gram-positive bacterium.

24. The method of claim 23, wherein the Gram-positive bacterium is elected from the group consisting of Staphylococcus aureus, Staphylococcus epidermidis, Staphylococcus haemolyticus, Staphylococcus hominis, Staphylococcus saprophyticus, Staphylococcus aureus, Streptococcus pneumoniae, Streptococcus pyogenes, Streptococcus agalactiae, Streptococcus dysgalactiae, Streptococcus avium, Streptococcus bovis, Streptococcus lactis, Streptococcus sangius, Streptococcus anginosus, Streptococcus intermedius, Streptococcus constellatus, Viridans streptococci, Enterococcus faecalis, Enterococcus faecium, Clostridium difficile, Clostridium clostridiiforme, Clostridium innocuum, Clostridium perfringens, Clostridium tetani, Mycobacterium tuberculosis, Mycobacterium avium, Mycobacterium intracellulare, Mycobacterium kansaii, Mycobacterium gordonae, Mycobacterium africanum, Mycobacterium bovis, Mycobacterium canetti, Mycobacterium caprae, Mycobacterium colombiense, Mycobacterium avium hominissuis, Mycobacterium microti, Mycobacterium mungi, Mycobacterium orygis, Mycobacterium pinnipedii, Mycobacterium avium silvaticum, Mycobacterium suricattae, Mycobacterium ulcerans, or Mycobacterium xenopi, Mycobacteria sporozoites, Listeria monocytogenes, Bacillus subtilis, Bacillus anthracis, Corynebacterium diphtherias, Corynebacterium jeikeium, Corynebacterium sporozoites, Erysipelothrix rhusiopathiae, and Actinomyces israelii.

25. The method according to any one of claim 19 to 24, wherein the compounds is administered in combination or alternation with an additional therapeutic agent selected from acedapsone, clofazimine, dapsone, desoxyfructo-serotonin, ethambutol, ethionamide, isoniazid, moxifloxacinor, pyrazinamide, rifapentine, streptomycin, sulfameter, thiacetazone, thalidomide, and combinations thereof.

26. The method according to any one of claim 19 to 25, wherein the subject is a mammal.

27. The method according to any one of claim 19 to 26, wherein the subject is a human.

28. The method according to any one of claim 19 to 27, wherein the subject is a nonhuman.

29. The method according to any one of claim 18 to 28, wherein the administering comprises topical administration, systemic administration, parenteral administration, subcutaneous administration, or transdermal administration, rectal administration, oral administration, intravaginal administration, intranasal administration, intrabronchial administration, intraocular administration, intra-aural administration, intravenous administration, intramuscular administration, or intraperitoneal administration.

30. A composition comprising the compound represented by any one of claim 1-12 or a salt, solvate or stereoisomer thereof and a excipient, carrier or diluent.

31. The composition according to claim 30, wherein the composition is a pharmaceutical composition and the carrier, excipient or diluent is a pharmaceutically acceptable carrier, excipient or diluent.

32. The composition according to claim 30, wherein the carrier, excipient or diluent is an agriculturally acceptable carrier, excipient or diluent.

33. The pharmaceutical composition according to claim 31, in a form suitable for topical administration, systemic administration, parenteral administration, subcutaneous administration, or transdermal administration, rectal administration, oral administration, intravaginal administration, intranasal administration, intrabronchial administration, intraocular administration, intra-aural administration, intravenous administration, intramuscular administration, or intraperitoneal administration.

34. The pharmaceutical composition according to claim 31 or 33, further comprising at least one additional therapeutic agent.

35. The composition according to any of claim 30 to 34, wherein the compound is obtained by culturing a microorganism having an ability to produce the compound in a nutrient medium.

36. The composition according to claim 35, wherein the microorganism is Photorhabdus australis strain DSM 17609.

37. A composition for combatting, controlling or inhibiting a pest, comprising a pesticidally effective amount of the compound according to any one of claim 1-12 or a salt, solvate or stereoisomer thereof and at least one agriculturally acceptable carrier, excipient or diluent.

38. The composition according to claim 37, in a form of topical administration, systemic administration, parenteral administration, subcutaneous administration, or transdermal administration, rectal administration, oral administration, intravaginal administration, intranasal administration, intrabronchial administration, intraocular administration, intra-aural administration, intravenous administration, intramuscular administration, or intraperitoneal administration.

39. The composition according to claim 37 or 38, wherein the compound is obtained by culturing a microorganism having an ability to produce the compound in a nutrient medium.

40. The composition according to any one of claim 37 to 39, wherein the microorganism is Photorhabdus australis strain DSM 17609.

41. A method of combatting, controlling or inhibiting a pest comprising exposing the pest to a pesticidally effective amount of the compounds according to any one of claim 1-12 or a salt, solvate or stereoisomer thereof.

42. The method of claim 41, wherein the pest is selected from an insect, a fungi, a bacteria, a nematode, a mite, or a tick.
